(19) United States
(12) Patent Application Publication
(10) Pub. No.: US 2026/0080972 A1
(43) Pub. Date: Mar. 19, 2026

(54) SYSTEMS AND METHODS FOR DISCOVERING COMPOUNDS USING HIERARCHICAL REINFORCEMENT LEARNING

(71) Applicant: DeepCure Inc., Boston, MA (US)

(72) Inventors: Derek Miller, Norfolk, MA (US); Jonathan Kaufman, Malden, MA (US); Matthew Tieman, Boston, MA (US)

(21) Appl. No.: 19/330,632

(22) Filed: Sep. 16, 2025

Related U.S. Application Data
(60) Provisional application No. 63/696,258, filed on Sep. 18, 2024.

(51) Int. Cl. G16B 15/30; G16B 5/00; G16C 20/10; G16C 20/50

(52) U.S. Cl. CPC G16B 15/30; G16B 5/00; G16C 20/10; G16C 20/50

(57) ABSTRACT

A method for identifying derived compounds exhibiting activity for a target macromolecule generates experiences. Each experience uses an initial compound in plurality of initial compounds to construct a derived compound through a hierarchical proximal policy. The policy has a parent molecular reaction model and a child reactant model that uses an environment of the target macromolecule. The parent model evaluates a plurality of molecular reactions. The child model evaluates a corresponding plurality of reactants for a selected molecular reaction. Using the plurality of experiences, the parameters of the parent model are updated in accordance with a first surrogate objective while the parameters of the child model are updated in accordance with a second surrogate objective. The generation of derived compounds and hierarchical proximal policy updating continues until convergence. Then, a subset of the derived compounds from the experiences is tested for activity against the target macromolecule.

### CROSS REFERENCE TO RELATED APPLICATIONS

This application claims priority to U.S. Provisional Patent Application No. 63/696,258 entitled “SYSTEMS AND METHODS FOR DISCOVERING COMPOUNDS USING HIERARCHICAL REINFORCEMENT LEARNING,” filed Sep. 18, 2024, which is hereby incorporated by reference.

### TECHNICAL FIELD

This application is directed to using hierarchical reinforcement learning to discover compounds that exhibit a threshold activity with respect to a target macromolecule.

### BACKGROUND

Pharmaceutical companies spend millions of dollars screening compounds to discover novel compounds and develop them into prospective drug leads. Traditionally, this has involved collecting large libraries of compounds tested to find the small number of compounds that interact with the disease target of interest. Unfortunately, gathering these large screening collections imposes significant challenges through storage constraints, shelf stability, or chemical cost. Furthermore, the cost and time needed to physically assay of compounds is prohibitive to testing them at scale. Even the largest pharmaceutical companies are testing only hundreds of thousands to a few millions of compounds at a time, versus the tens of millions of commercially available compounds and the billions, and even trillions of compounds that can be generated and screened computationally.

One key characteristic of a successful drug candidate is strong binding against its disease target. However, compounds that bind strongly enough to be clinically effective are rare.

Approximately half of the drug candidates in late-stage clinical trials fail due to unacceptable toxicity. Toxicity can be due to off-target side effects caused by a compound binding non-selectively to other targets. Therefore, increasing potent binding to the desired target while decreasing non-selective binding to other related targets is important in drug discovery. Drug candidates can also fail because they do not have desirable pharmacological absorption, distribution, metabolic, and excretion (ADME) profiles. Optimizing and balancing multiple objectives such as potency, selectivity, toxicity, and pharmacological properties is challenging but essential for a compound to become a drug.

Due to the many requirements for a compound to be a drug, there is a need to explore large and diverse chemical spaces of compounds that have different interactions with the target and, therefore, different properties. Large and diverse libraries of compounds also increase the odds of finding compounds that simultaneously satisfy all the other ADME properties needed to be a safe and effective drug. Thus, a better method is needed to accurately, rapidly, and efficiently identify or generate compounds that interact with the desired target.

Given the above background, what is needed in the art are methods for designing, identifying, and/or generating candidate compounds having target interaction properties when complexed with target macromolecules.

### SUMMARY

The present disclosure addresses the problems identified in the background by providing systems and methods that identify derived compounds exhibiting activity for a target macromolecule by generating experiences. Each experience starts with an initial compound selected from a plurality of initial compounds. A hierarchical proximal policy is applied to the initial compound of an experience resulting in successive chemical modifications of the initial compound over a series of states. That is, at each state, the initial compound, in its predecessor state, is chemically modified. The policy has a parent molecular reaction model and a child reactant model that uses an environment of the target macromolecule at each state. At each state, the parent model evaluates the suitability of a plurality of molecular reactions for the initial compound in the given state in the context of the environment of the target macromolecule. The parent model gives each molecular reaction a probability. Then, one of the molecular reactions is selected (sampled) based on this probability assignment. The child model evaluates a corresponding plurality of reactants for the molecular reaction that was selected from the sampling process. The process of evolving an initial compound continues until an experience exit condition is satisfied. Where there are a sufficient number of experiences, the parameters of the parent model are updated in accordance with a first surrogate objective while the parameters of the child model are updated in accordance with a second surrogate objective. The generation of derived compounds and hierarchical proximal policy updating continues until convergence. Then, a subset of the derived compounds from the experiences is tested in a wet lab assay for activity against the target macromolecule.

In more detail, one aspect of the present disclosure provides a method for identifying one or more derived compounds that exhibit a threshold activity with respect to a target macromolecule, using a plurality of initial compounds.

In some embodiments, the target macromolecule is a protein, a polypeptide, a polynucleic acid, a polyribonucleic acid, a polysaccharide, or an assembly of any combination thereof.

In some embodiments, each initial compound 210 in the plurality of initial compounds is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Dalton, or less than 1000 Daltons. In some embodiments, each initial compound 210 in the plurality of initial compounds is an organic compound having a molecular weight of between 500 Daltons and 1000 Daltons.

In some embodiments, each initial compound 210 in the plurality of initial compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, the plurality of initial compounds comprises 100 or more, 500 or more, 1000 or more, 2000 or more, 10,000 or more, 100,000 or more, 1×10⁶ or more, 1×10⁷ or more, or 1×10⁸ or more initial compounds.

### Generating a Plurality of Experiences.

In the present disclosure, a plurality of experiences is generated. Each respective experience in the plurality of experiences using an initial compound selected from the plurality of initial compounds to construct a corresponding derived compound through a hierarchical proximal policy comprising a parent (molecular reaction) model and a child (reactant) model using an environment of the target macromolecule, thereby generating a plurality of derived compounds.

In some embodiments, the environment of the target macromolecule is a binding pocket of the target macromolecule.

In some embodiments, the environment of the target macromolecule is defined by a plurality of atomic coordinates of atoms of residues of the binding pocket derived by X-ray crystallography, neutron diffraction, cryo-electron microscopy, sampling from computational simulations, homology modeling, rotamer library sampling, or any combination thereof.

In some embodiments, each derived compound in the plurality of derived compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. In some embodiments, each derived compound in the plurality of derived compounds is an organic compound having a molecular weight of between 400 Daltons and 10000 Daltons.

In some embodiments, each derived compound in the plurality of derived compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, the parent model is a molecular reaction model that evaluates a plurality of molecular reactions, and the child model is a reactant model that evaluates a corresponding plurality of reactants for a molecular reaction.

In some embodiments, the parent model is a first graph neural network (e.g., a first graph isomorphism neural network).

In some embodiments, the child model is a second graph neural network (e.g., a second graph isomorphism neural network) that is passed an output of the parent model.

In some embodiments, the parent model comprises a first plurality of parameters (e.g., at least 10,000, at least 100,000, or at least 1×10⁶ parameters), and the child model comprises a second plurality of parameters (e.g., at least 10,000, at least 100,000, or at least 1×10⁶ parameters).

In some embodiments, the plurality of molecular reactions comprises named reactions, organic synthesis reactions or protecting group reactions.

In some embodiments, the corresponding plurality of reactants is a corresponding plurality of synthons. In some embodiments, the corresponding plurality of reactants comprises twenty or more reactants. In some embodiments, the corresponding plurality of reactants comprises 20 or more synthons, 50 or more synthons, 100 or more synthons, 1000 or more synthons, 10,000 or more synthons, 100,000 or more synthons, or 1×10⁶ or more synthons.

In some embodiments, an experience in the plurality of experiences is generated by:
(i) Initializing the experience to state t=0;
(ii) inputting a complex of state t, in two or three dimensions, of the initial compound in state t interacting with the environment of the target macromolecule into the parent model 184. The parent model evaluates a first exit vector of the initial compound in state t against the plurality of molecular reactions, thereby assigning a corresponding probability to each respective molecular reaction in the plurality of molecular reactions for state t.
(iii) Selecting a molecular reaction in the plurality of molecular reactions, through a sampling of the plurality of molecular reactions using the corresponding probability assigned to each molecular reaction in the plurality of molecular reactions for state t.
(iv) Inputting the complex of state t into the child model. The child model evaluates the initial compound in state t against each reactant in a corresponding plurality of reactants available for reaction using the molecular reaction selected for state t, thereby assigning a corresponding probability to each respective reactant in the corresponding plurality of reactants for state t.
(v) Selecting a reactant in the corresponding of plurality of reactants, through a sampling of the corresponding plurality of reactants using the corresponding probability assigned to each reactant in the corresponding plurality of reactants for state t.
(vi) Advancing state t to state t+1.
(vii) Forming the initial compound in state t through an in silico reaction of the initial compound in state t−1 in accordance with the selected molecular reaction 212 and the selected reactant 214 of state t.
(viii) Determining a score for the initial compound 210 in state t interacting with the environment of the target macromolecule by inputting the initial compound in state t interacting with the environment of the target macromolecule into a physics model.
(ix) Repeating (ii), (iii), (iv), (v), (vi), (vii), and (viii) until a compound exit criterion (e.g., the compound exit criterion comprises a molecular weight, a molecular weight range, a logₚ, or a logₚ range) is satisfied by the initial compound in state t, thereby forming a plurality of states for the experience. In some embodiments, the initial compound in state t is assigned a terminal positive reward when the compound exit criterion is satisfied. In some embodiments, the initial compound in state t is assigned a terminal negative reward when the compound exit criterion is satisfied.

In some embodiments, the compound exit criterion is satisfied by either a negative condition of the initial compound in state t or a positive condition of the initial compound in state t. When the initial compound in state t has the positive condition, a terminal positive reward is assigned to the initial compound in state t and the (ix) repeating is terminated. When the initial compound in state t has the negative condition, a terminal negative reward is assigned to the initial compound in state t and the (ix) repeating is terminated.

In some embodiments, the first surrogate objective is a first trust region method.

In some embodiments, the first surrogate objective is a clipped surrogate objective.

In some embodiments, the physics model evaluates an interaction energy of a complex of the initial compound in state t interacting with the environment of the target macromolecule.

In some embodiments, the physics model evaluates an interaction energy of a complex of the initial compound in state t interacting with the environment of the target macromolecule using a calculated potential energy surface of the initial compound and the environment of the target macromolecule.

In some such embodiments, the potential energy surface is calculated by the physics model using a molecular mechanics algorithm.

In some such embodiments, the potential energy surface is calculated by the physics model using a quantum mechanics algorithm.

In some embodiments, the physics model evaluates the initial compound in state t interacting with the environment of the target macromolecule against an interaction feature contract.

In some embodiments, a derived compound in the corresponding plurality of derived compounds requires at least two, at least three, or at least four different molecular reactions in the plurality of molecular reactions to be synthesized from an initial compound in state t=0 used by the method to construct the derived compound.

In some embodiments, the complex of the initial compound in state t interacting with the environment 154 of the target macromolecule 152 comprises a plurality of poses (e.g., 2 or more poses, 10 or more poses, 100 or more poses, or 1000 or more poses) of the initial compound in state t docked into the environment of the target macromolecule.

In some embodiments, the plurality of molecular reactions comprises twenty or more molecular reactions, or one hundred or more molecular reactions.

In some embodiments, the method further comprises masking those molecular reactions in the plurality of molecular reactions that are incompatible with an exit vector in an initial compound.

In some embodiments, the plurality of experiences is twenty or more experiences representing 20 or more initial compounds in the plurality of initial compounds.

### Updating the Policy.

When there are a sufficient number of experiences, the first plurality of parameters of the parent model is updated in accordance with a first surrogate objective calculated using the plurality of experiences. Further, the second plurality of parameters of the child model 186 is updated in accordance with a second surrogate objective 190 using the plurality of experiences.

The process of generating derived compounds in the experiences, and updating the parent and child models is repeated until a threshold convergence criterion is satisfied.

In some embodiments a subset of the plurality of derived compounds, from the plurality of experiences, is tested in an assay (e.g., a wet lab assay) for activity against the target macromolecule, thereby identifying one or more derived compounds that exhibit the threshold activity with respect to the target macromolecule.

In some embodiments, the threshold activity with respect to the target macromolecule is an IC₅₀, EC₅₀, K_{d}, K_{I}, hill coefficient (nH), negative logarithm of EC₅₀ (pEC₅₀), association rate constant (Kon), or disassociation rate constant (Koff), for a derived compound with respect to the target macromolecule.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIG. 1 illustrates a computer system in accordance with some embodiments of the present disclosure.

FIGS. 2A and 2B collectively illustrate data structures in accordance with some embodiments of the present disclosure.

FIG. 3 is a schematic view of an example workflow for identifying one or more derived compounds that exhibit a threshold activity with respect to a target macromolecule, in accordance with some embodiments of the present disclosure.

FIGS. 4A, 4B, and 4C illustrate an example workflow for identifying one or more derived compounds that exhibit a threshold activity with respect to a target macromolecule, in accordance with some embodiments of the present disclosure.

FIG. 5 illustrates an initial compound at various states within an experience, culminating in a derived compound, in accordance with some embodiments of the present disclosure.

FIGS. 6A, 6B, 6C, 6D, 6E, 6F, 6G, 6H, and 6I is a detailed flowchart for identifying one or more derived compounds that exhibit a threshold activity with respect to a target macromolecule, in accordance with some embodiments of the present disclosure, in which optional elements of the flowchart are indicated by dashed boxes.

FIG. 7 illustrates an example representation of a transformed interaction feature vector, in which values for interaction features are binarized, in accordance with some embodiments of the present disclosure.

FIG. 8 illustrates a parent and child model using in accordance with an embodiment of the present disclosure.

FIG. 9 illustrates a stylized view of a target macromolecule with an environment that is a binding pocket in accordance with the prior art.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Drug discovery efforts often suffer from significant bottlenecks, including the ability to identify hit compounds and validate any such identified hit compounds as lead compounds for eventually synthesis and testing. These difficulties can be attributed, at least in part, to the massive size of custom molecule libraries that are searched in these early stages, which can reach up to 10¹² candidate molecules. Conventional methods, including traditional screening, fragment-based screening, and various machine learning and artificial intelligence pipelines, require laborious hit identification and/or hit-to-lead steps that increase the overall time, cost, and resource expenditure of drug discovery.

Advantageously, the systems and methods disclosed herein allow for rational design of molecules that meet stringent criteria, such binding, selectivity, and/or pharmacological requirements using hierarchical reinforcement learning. In particular, the systems and methods disclosed herein provide a unique platform that can be used to identify lead-like candidate in ultra-large custom libraries for target macromolecules.

The present disclosure identifies derived compounds exhibiting activity for a target macromolecule by generating experiences. Each experience starts with an initial compound selected from a plurality of initial compounds. The initial compound of an experience is evolved through a series of stages. At each stage, the initial compound is chemically modified using a hierarchical set of models, comprising a parent model that is used to first select the molecular reaction to be applied to the initial compound on a probabilistic basis. When the selected molecular reaction requires a reactant, a child model is used that, based on the identity of the selected reaction, selects the reactant to be used with the selected molecular reaction to chemically modify the initial compound.

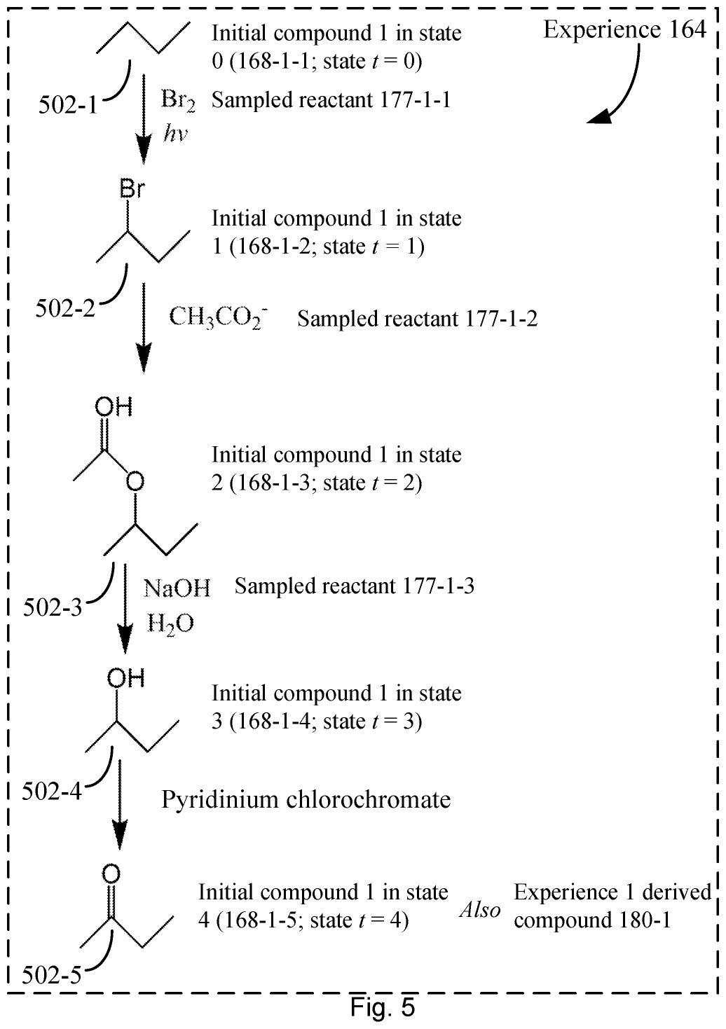

For example, in FIG. 5, at state t=0, the initial compound has structure 502-1. A halogenation reaction is selected for state t=0 through the use of the parent model, and bromine is selected through the use of the child model resulting in the initial compound at state t=1 having structure 502-2.

A substitution reaction is selected for state t=1 through the use of the parent model, and acetate is selected through the use of the child model resulting in the initial compound at state t=2 having structure 502-3.

A hydrolysis reaction is selected for state t=2 through the use of the parent model. This is a unimolecular reaction and thus the child model is not used to select a reactant for state t=2. The result of the in silico reaction of state t=2 is the initial compound at state t=3, which has now been hydrolyzed to have the structure 502-4.

An oxidation reaction is selected for state t=3 through the use of the parent model. This is a unimolecular reaction and thus the child model is not used to select a reactant for state t=3. The result of the in silico reaction selected for state t=3 is the initial compound at state t=4, which has now been oxidized to have the structure 502-5.

The initial structure 502-5 at state t=4 satisfies an exit condition and thus is assigned the derived structure for the experience.

Thus, as illustrated in FIG. 5, the successive chemical modifications of the initial compound over a series of states results in a final derived structure. As illustrated in FIG. 5, at each state t, the initial compound, in its predecessor state, is chemically modified. At each state t, the parent molecular reaction model and, when needed, the child reactant model uses an environment of the target macromolecule at that state t to identify molecular reactions, and for reactions other than unimolecular reactions, a reactant. Thus, at each state, the parent model evaluates the suitability of a plurality of molecular reactions for the initial compound in the given state in the context of the environment of the target macromolecule. The parent model gives each molecular reaction a probability. Then, one of the molecular reactions is selected (sampled) based on this probability assignment. In cases where the reaction involves more than a single reactant (e.g., more than just the initial compound in state t), the child model evaluates a corresponding plurality of reactants for the molecular reaction that was selected from the parent model sampling process. The process of evolving an initial compound as illustrated in FIG. 5 continues until an experience exit condition is satisfied.

When there are a sufficient number of experiences, the parameters of the parent model are updated in accordance with a first surrogate objective while the parameters of the child model are updated in accordance with a second surrogate objective.

The generation of derived compounds in experiences and hierarchical proximal policy updating continues until convergence.

Then, a subset of the derived compounds from the experiences is tested in a wet lab assay for activity against the target macromolecule.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

### Definitions

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “target” refers to an object of interest, such as a macromolecule, macromolecule complex, or polymer that is of interest as a primary binding target for a candidate molecule. As used herein, the term “off-target” refers to an object that is not the primary binding target, such as a macromolecule, macromolecule complex, or polymer that exhibits off-target binding with a candidate molecule.

As used interchangeably herein, the terms “pose” or “conformation” refer to a pose of a compound when complexed to a target macromolecule. In some embodiments, a pose refers to the complex formed between a target macromolecule and any suitable compound capable of complexing to the target macromolecule including, but not limited to a initial compound, derived compound, a ligand, a reference molecule, a training molecule, a molecular component, and/or a molecular intermediate.

In some embodiments, a pose is determined by one or more docking programs. In some embodiments, one docking program is used to determine some of the poses for a complex between a compound and a target macromolecule and another docking program is used to determine other poses for the complex between the compound and the target macromolecule.

In some embodiments, one or more poses are determined using AutoDock Vina. See, Trott and Olson, “AutoDock Vina: improving the speed and accuracy of docking with a new scoring function, efficient optimization and multithreading,” Journal of Computational Chemistry 31 (2010) 455-461. In some embodiments, one or more poses are determined using Quick Vina 2 (Alhossary et al., 2015, “Fast, accurate, and reliable molecular docking with Quick Vina,” Bioinformatics 31:13, pp. 2214-2216), VinaLC (Zhang et al., 2013, “Message Passing Interface and Multithreading Hybrid for Parallel Molecular Docking of Large Databases on Petascale High Performance Computing Machines,” J. Comput. Chem. DOI: 10.1002/jcc.23214), Smina (Koes et al., 2013, “Lessons learned in empirical scoring with smina from the CSAR 2011 benchmarking exercise,” Journal of chemical information and modeling 53:8, pp. 1893-1904), or CUina (Morrison et al., “Efficient GPU Implementation of AutoDock Vina,” COMP poster 3432389).

In some embodiments, one or more ensembled poses are determined using an ensembled docking algorithm such as disclosed in Stafford et al., 2022, “AtomNet PoseRanker: Enriching Ligand Pose Quality for Dynamic Proteins in Virtual High-Throughput Screens,” Journal of Chemical Information and Modeling 62, pp. 1178-1189, which is hereby incorporated by reference. In some such embodiments the ensemble consists of between 3 and 64, between 4 and 128, between 5 and 32, more than 5, or between 8 and 25 structurally similar poses.

In some embodiments, a compound is docked to a target macromolecule by either random pose generation techniques or by biased pose generation. In some embodiments, a compound is docked to a macromolecule by Markov chain Monte Carlo sampling. In some embodiments, such sampling allows the full flexibility of the compound in the docking calculations and a scoring function that is the sum of the interaction energy between the compound and the macromolecule as well as the conformational energy of the molecule. See, for example, Liu and Wang, 1999, “MCDOCK: A Monte Carlo simulation approach to the molecular docking problem,” Journal of Computer-Aided Molecular Design 13, 435-451, which is hereby incorporated by reference.

In some embodiments, algorithms such as DOCK (Shoichet, Bodian, and Kuntz, 1992, “Molecular docking using shape descriptors,” Journal of Computational Chemistry 13(3), pp. 380-397; and Knegtel et al., 1997 “Molecular docking to ensembles of protein structures,” Journal of Molecular Biology 266, pp. 424-440, each of which is hereby incorporated by reference) are used to find the one or more poses for a compound against a target macromolecule. Such algorithms model the macromolecule and the compound as rigid bodies. The docked conformation is searched using surface complementary to find poses.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference); FlexX (Rarey et al., 1996, “A Fast Flexible Docking Method Using an Incremental Construction Algorithm,” Journal of Molecular Biology 261, pp. 470-489, which is hereby incorporated by reference); GOLD (Jones et al., 1997, “Development and Validation of a Genetic Algorithm for flexible Docking,” Journal Molecular Biology 267, pp. 727-748, which is hereby incorporated by reference) are used to find one or more poses.

In some embodiments, molecular dynamics is performed on a target macromolecule (or a portion thereof such as the active site of the macromolecule) and a compound to identify one or more poses for the compound. During the molecular dynamics run, the atoms of the macromolecule and compound are allowed to interact for a fixed period of time, giving a view of the dynamical evolution of the system. In some embodiments, the trajectory of atoms in the target macromolecule and the compound are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,” J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the macromolecule and the compound (e.g., initial compound, derived compound, etc.) over time. This trajectory comprises the trajectory of the atoms in the target macromolecule and the compound. In some embodiments, a subset of the plurality of different poses is obtained by taking snapshots of this trajectory over a period of time. In some embodiments, poses are obtained from snapshots of several different trajectories, where each trajectory comprises a different molecular dynamics run of the target macromolecule interacting with the compound. In some embodiments, prior to a molecular dynamics run, the compound is first docked into an active site of the target macromolecule using a docking technique.

As used herein, the term “parameter” refers to any coefficient or, similarly, any value of an internal or external element (e.g., a weight and/or a hyperparameter) in a model, regressor, and/or classifier that affects (e.g., modify, tailor, and/or adjust) one or more inputs, outputs, and/or functions in the model, regressor and/or classifier. For example, in some embodiments, a parameter refers to any coefficient, weight, and/or hyperparameter that is used to control, modify, tailor, and/or adjust the behavior, learning and/or performance of a model, regressor, and/or classifier. In some instances, a parameter is used to increase or decrease the influence of an input (e.g., a feature) to a model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given model, regressor, and/or classifier but can be used in any suitable model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for a model, regressor, and/or classifier (e.g., by error minimization and/or backpropagation methods, as described elsewhere herein).

In some embodiments, a model, regressor, and/or classifier of the present disclosure comprises a plurality of parameters. In some embodiments the plurality of parameters is n parameters, where n is an integer and n≥2, n≥5, n≥10, n≥25, n≥40, n≥50, n≥75, n≥100, n≥125, n≥150, n≥200, n≥225, n≥250, n≥350, n≥500, n≥600, n≥750, n≥1,000, n≥2,000, n≥4,000, n≥5,000, n≥7,500, n≥10,000, n≥20,000, n≥40,000, n≥75,000, n≥100,000, n≥200,000, n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and Is that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

As used herein, the term “graph neural network” (GNN) refers to a model that is suitable for representation learning of graphs. A GNN follow a neighborhood aggregation scheme, where the representation vector of a node is computed by recursively aggregating and transforming representation vectors of its neighboring nodes. After k iterations of aggregation, a node is represented by its transformed feature vector, which captures the structural information within the node's k-hop neighborhood. The representation of an entire graph can then be obtained through pooling, for example, by summing the representation vectors of all nodes in the graph. Input to a GNN includes molecular graphs, labeled graphs where the vertices and edges represent the atoms and bonds of the molecule, respectively. Graph neural networks and molecular graphs are further described, for example, in Xu et al., “How powerful are graph neural networks?” ICLR 2019, arXiv:1810.00826v3, which is hereby incorporated herein by reference in its entirety.

GNN variants for both node and graph classification tasks are known in the art. For example, in some embodiments, the first model is a graph convolutional neural network. Nonlimiting examples of graph convolutional neural networks are disclosed in Behler Parrinello, 2007, “Generalized Neural-Network Representation of High Dimensional Potential-Energy Surfaces,” Physical Review Letters 98, 146401; Chmiela et al., 2017, “Machine learning of accurate energy-conserving molecular force fields,” Science Advances 3 (5): e1603015; Schütt et al., 2017, “SchNet: A continuous-filter convolutional neural network for modeling quantum interactions,” Advances in Neural Information Processing Systems 30, pp. 992-1002; Feinberg et al., 2018, “PotentialNet for Molecular Property Prediction,” ACS Cent. Sci. 4, 11, 1520-1530; and Stafford et al., “AtomNet PoseRanker: Enriching Ligand Pose Quality for Dynamic Proteins in Virtual High Throughput Screens,” chemrxiv.org/engage/chemrxiv/article-details/614b905e39cf6a1c36268003, each of which is hereby incorporated by reference.

### Example Systems for Identifying One or More Derived Compounds that Exhibit a Threshold Activity with Respect to a Target Macromolecule

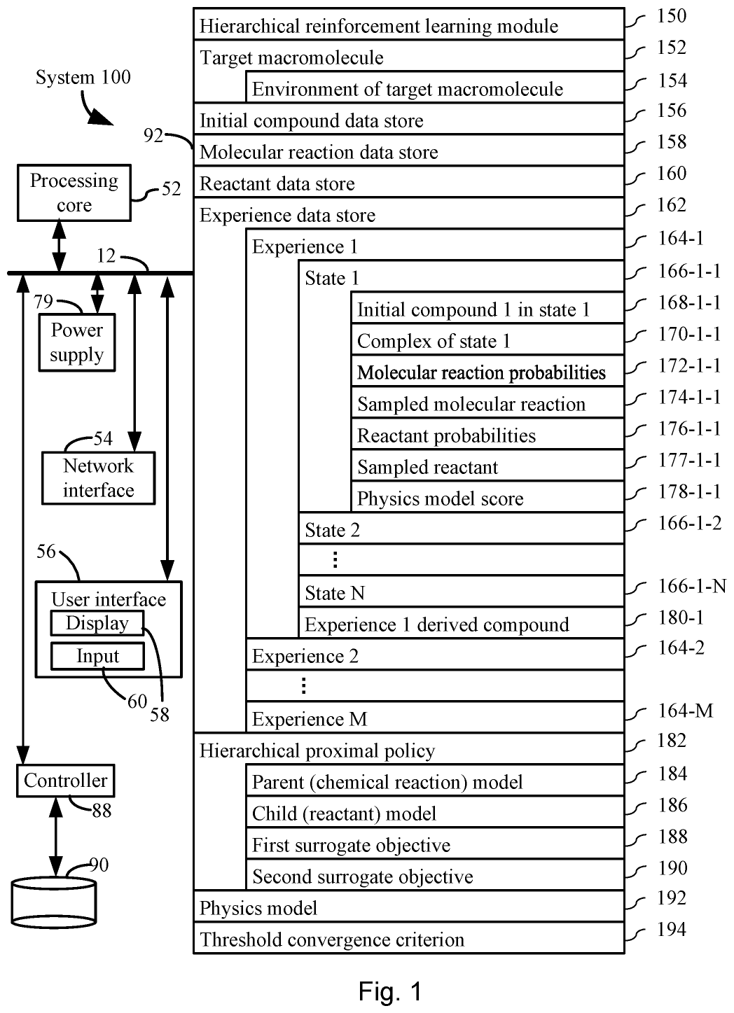

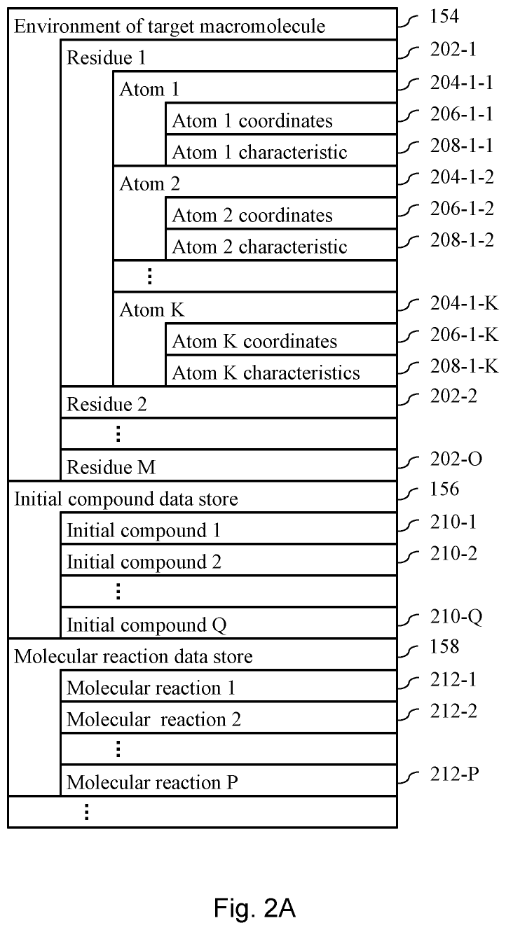

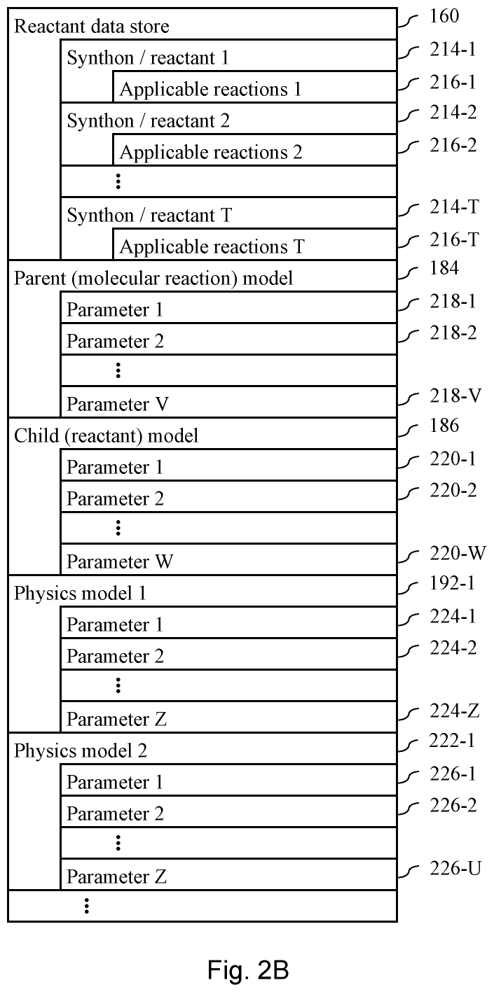

FIGS. 1, 2A, and 2B collectively illustrate a computer system 100 for identifying one or more derived compounds that exhibit a threshold activity with respect to a target macromolecule. Referring to FIGS. 1, 2A, and 2B in typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 can be spread across any number of networked computers and/or reside on each of several networked computers and/or virtual machines. One of skill in the art will appreciate that a wide array of different computer topologies is possible for the computer system 100 and all such topologies are within the scope of the present disclosure.

Turning to FIGS. 1, 2A, and 2B with the foregoing in mind, the computer system 100 comprises one or more processing units (CPUs, processing cores) 52, a network or other communications interface 54, a user interface 56 (e.g., including an optional display 58 and optional keyboard 60 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 52. In other words, some data stored in memory 92 and/or memory 90 may in fact be hosted on computers that are external to computer system 100 but that can be electronically accessed by the computer system 100 over an Internet, intranet, or other form of network or electronic cable using network interface 54. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

In some embodiments, the memory 92 of the computer system 100 stores:
Optional operating system (not shown in FIG. 1) that includes procedures for handling various basic system services;
Reinforcement learning module 150 for identifying one or more derived compounds that exhibit a threshold activity with respect to a target macromolecule;
A target macromolecule 152 comprising an environment of the target macromolecule 154 optionally defined by a plurality of residues 202-1, 202-2, . . . , 202-O, where O is a positive integer, and for each respective residue in the plurality of residues, one or more atoms (e.g., 204-1-1, 204-1-2, . . . , 204-1-K, where K is a positive integer) of the respective residue, and for each such atom, atom coordinates (e.g., coordinates 206 (e.g., 206-1-1, 206-1-2, . . . , 206-1-K) and characteristics 208 (e.g., 208-1-1, 208-1-2, . . . , 208-1-K);
Initial compound data store 156 comprising initial compounds 210-1, 210-2, . . . , 210-Q, where Q is a positive integer;
Molecular reaction data store 158 comprising molecular reactions 212-1, 212-2, . . . , 212-P, where P is a positive integer;
Reactant data store 160 comprising synthon/reactants 214-1, 214-2, . . . , 214-T, where T is a positive integer, and for each such synthon/reactant 214, an indication 216 of the applicable molecular reactions 212 for the synthon/reactant;
Experience data store 162 comprising experiences 164-1, 164-2, . . . 164-M, each such experience 164 comprising a plurality of states 166 and a final derived compound 180, each respective state 166 comprising the molecular structure of an initial compound 168 in the respective state, a description of the complex of the an initial compound 168 in the respective state with the environment 154 of the target macromolecule 152, a set of molecular reaction probabilities 172-1-1 for the molecular reactions 212, a selected (sampled) molecular reaction 174, an optional set of reactant probabilities 176 for those reactants that can be used with the selected (sampled) molecular reaction, an optional selected (sampled) reactant 177 for selected (sampled) molecular reaction, and a physics model score 178 for the complex of the an initial compound 168 in the respective state with the environment 154 of the target macromolecule 152;
Hierarchical proximal policy 182 comprising a parent (chemical reaction) model 184 with parameters 218-1, 218-2, . . . , 218-V, where V is a positive integer, and child (reactant) model 186 with parameters 220-1, 220-2, . . . , 220-W, where W is a positive integer, a first surrogate objective 188 for parent model 184 and a second surrogate objective 190 for child model 186;
Physics model 192-1 with parameters 224-1, 224-2, . . . , 224-Z, where Z is a positive integer;
Physics model 192-2 with parameters 226-1, 226-2, . . . , 226-U, where U is a positive integer; and
Threshold convergence criterion 194.

In some implementations, any two or more of N, M, K, O, Q, P, T, V, W, Z, and U are the same or a different positive integer value. In some embodiments N, M, K, O, Q, P, T, V, W, Z, or U is a positive integer (e.g., 2, 3, 4, 5, 6, 7, 8, 9, 10, 11, 12, 13, 14, 15, 16, 17, 18, 19, or 20 or more). In some embodiments N, M, K, O, Q, P, T, V, W, Z, or U is a positive integer that is at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹. In some embodiments, N, M, K, O, Q, P, T, V, W, Z, or U is a positive integer of no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000. In some embodiments, N, M, K, O, Q, P, T, V, W, Z, or U is a positive integer that is between 1000 and 100,000, 10,000 and 1×10⁷, 1×10⁶ and 1×10⁸, 1×10⁸ and 1×10¹¹, or 1×10⁹ and 1×10¹². In some embodiments, N, M, K, O, Q, P, T, V, W, Z, or U is a positive integer that falls within another range starting no lower than 10 and ending no higher than 1×10¹².

In some implementations, one or more of the above identified data elements or modules of the computer system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 optionally stores a subset of the modules and data structures identified above. Furthermore, in some embodiments the memory 92 and/or 90 stores additional modules and data structures not described above.

Methods for Identifying One or More Derived Compounds that Exhibit a Threshold Activity with Respect to a Target Macromolecule.

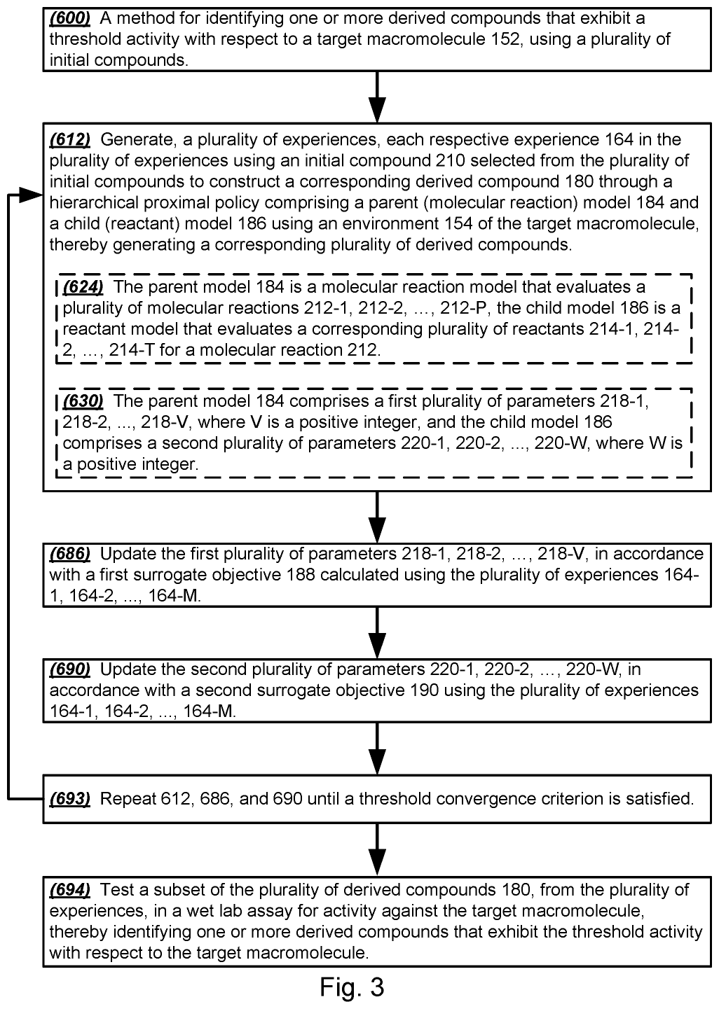

Now that a system for identifying one or more derived compounds that exhibit a threshold activity with respect to a target macromolecule has been described in conjunction with FIGS. 1, 2A, and 2B, an overview of a method for performing such identification is detailed with reference to FIGS. 3 and 5.

FIG. 3 provides a summary of one such method for identifying one or more derived compounds that exhibit a threshold activity with respect to a target macromolecule 152, using a plurality of initial compounds (block 600). FIG. 3 makes use of a hierarchical reinforcement learning approach. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety.

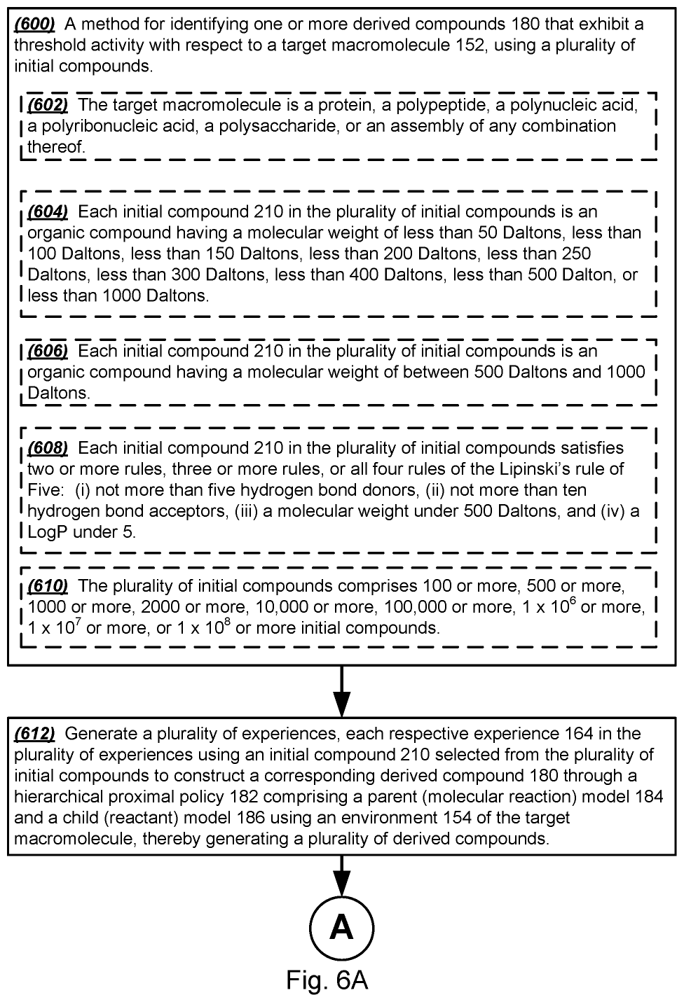

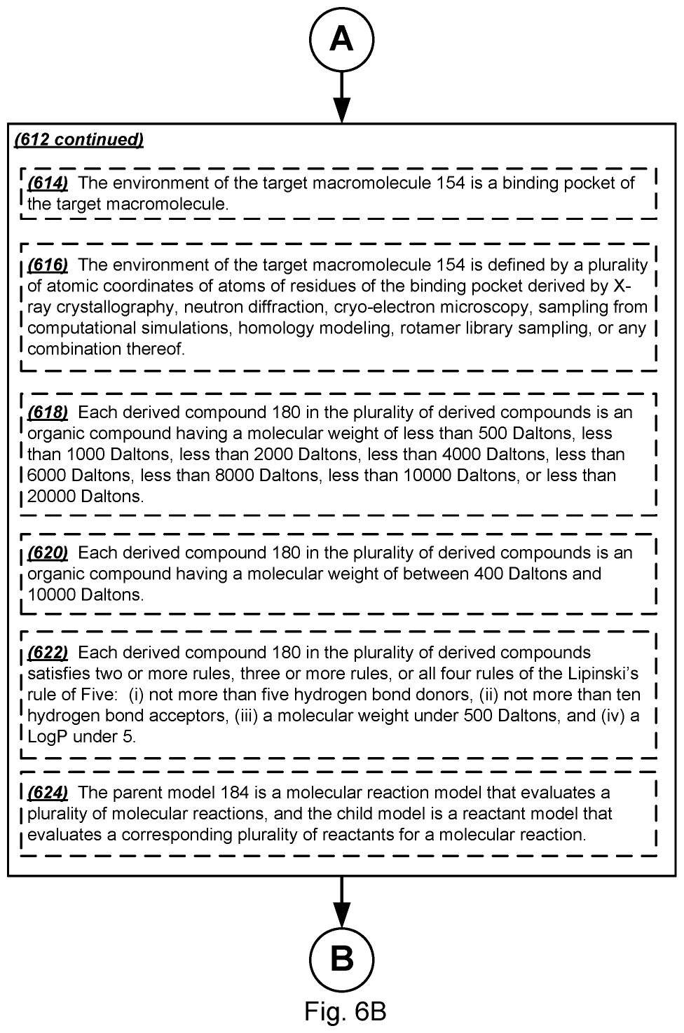

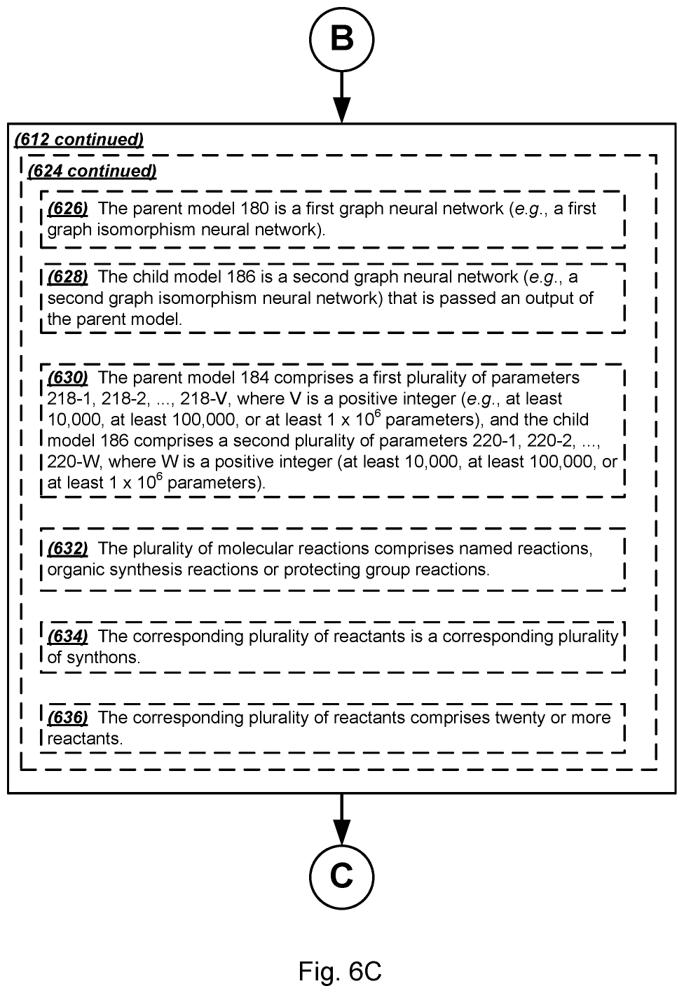

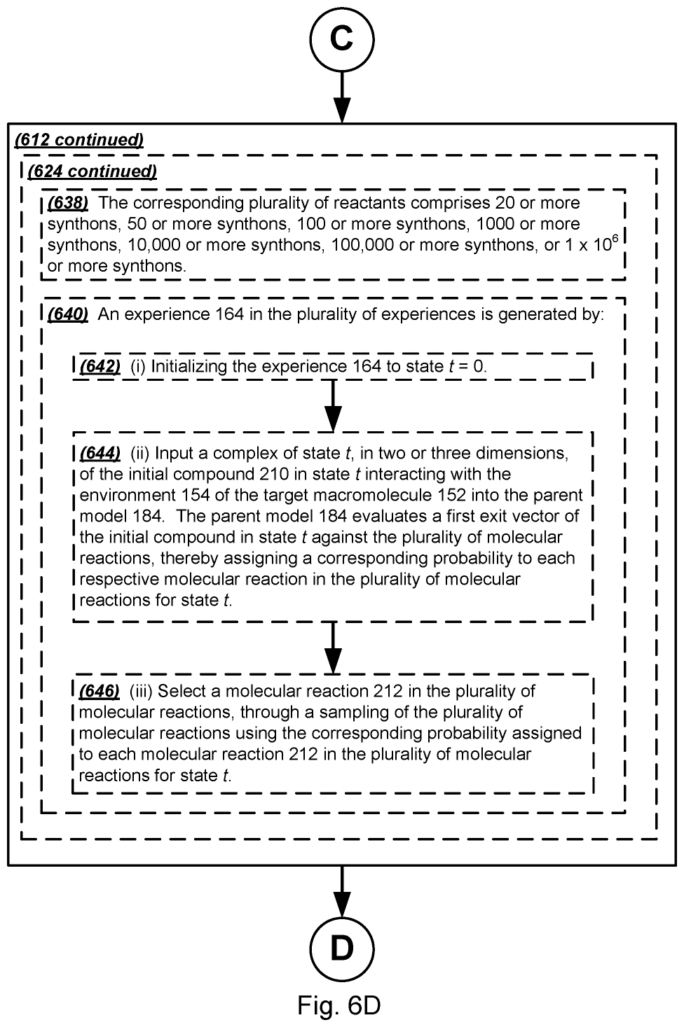

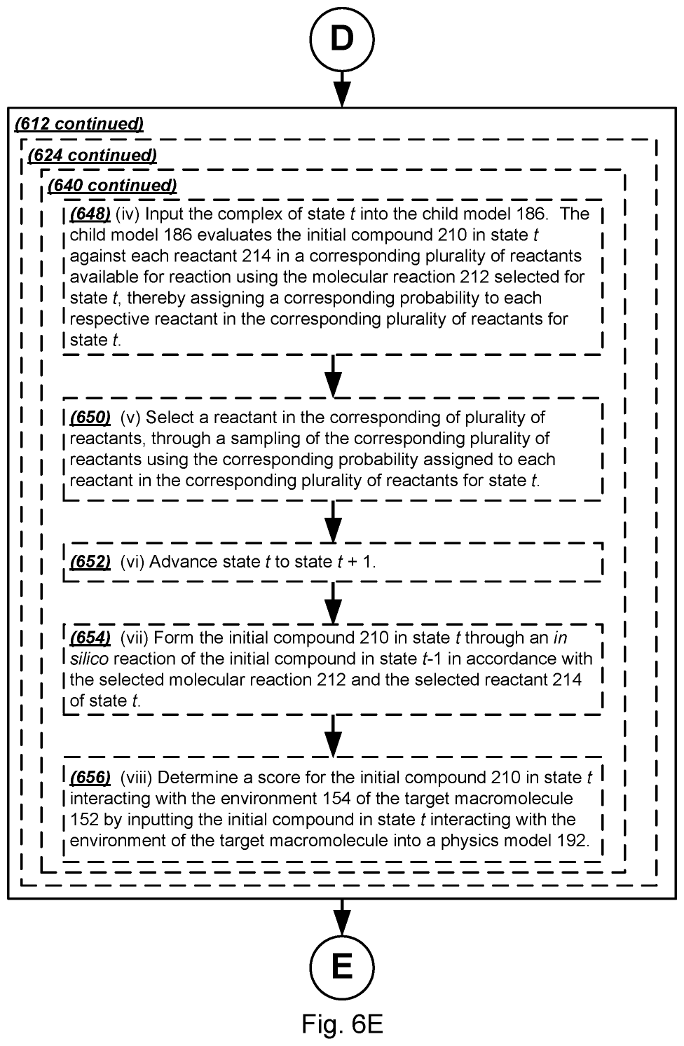

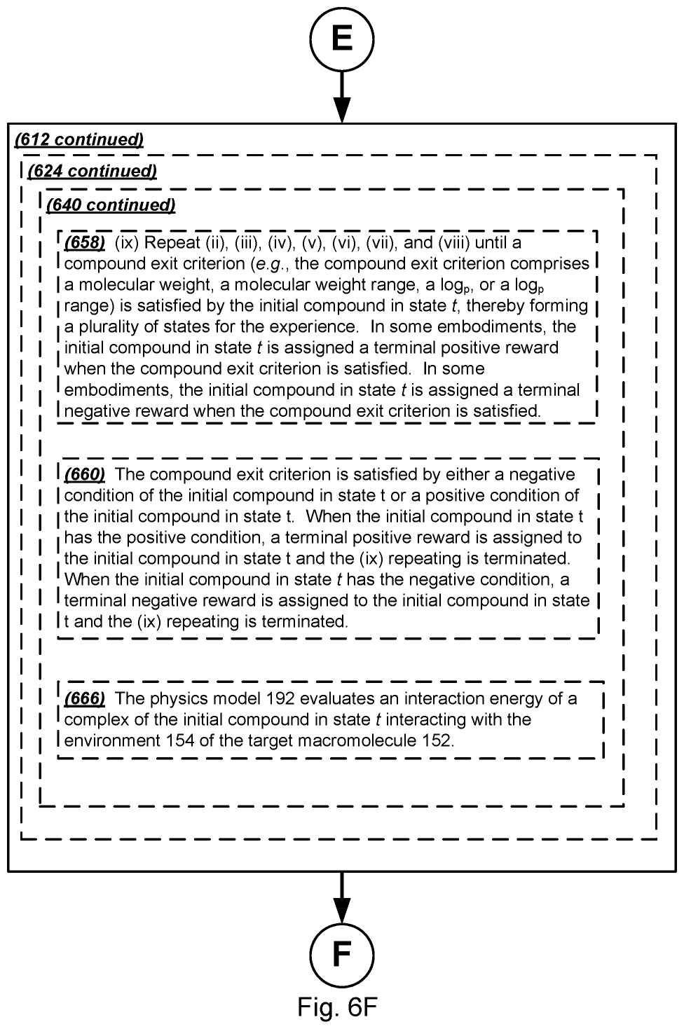

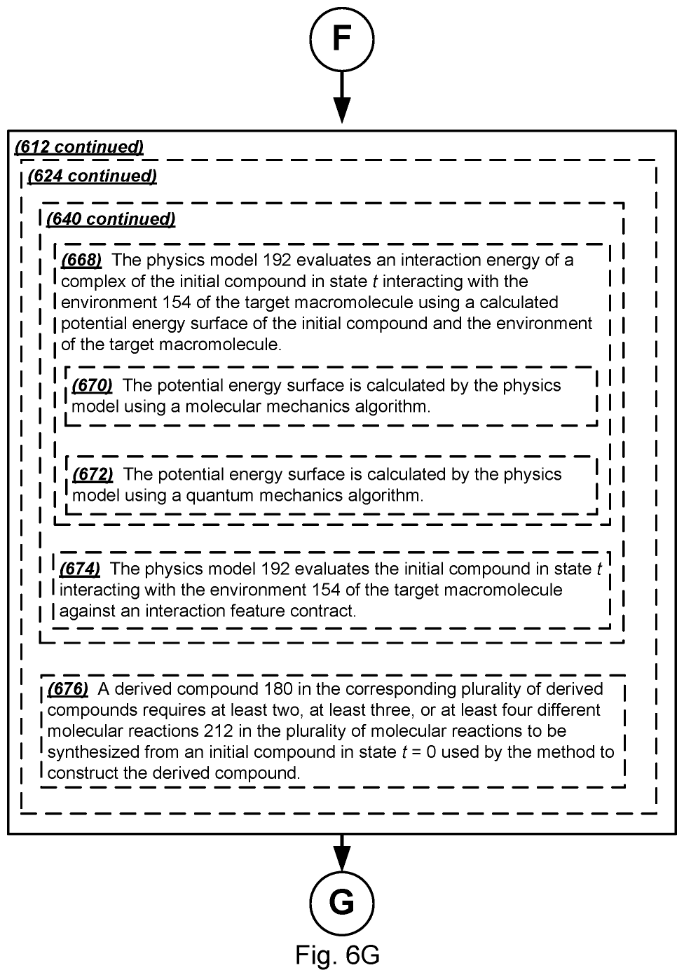

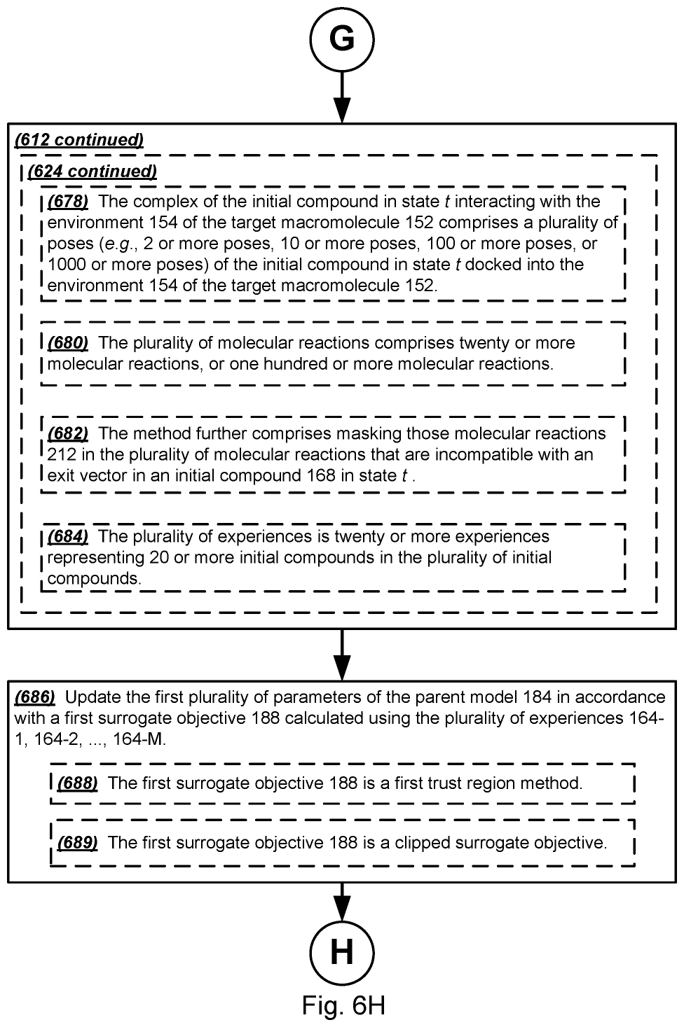

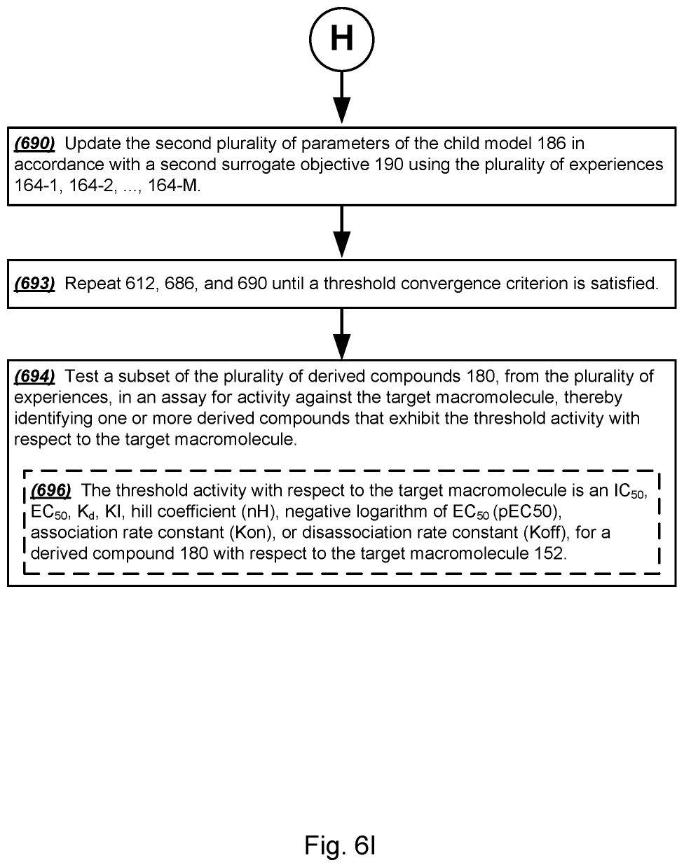

In accordance with block 612 of FIG. 3, described in further detail below in conjunction with the description of FIG. 6, a plurality of experiences is generated. One such experience is illustrated in FIG. 5. Each respective experience 164 in the plurality of experiences uses an initial compound 210 selected from a plurality of initial compounds to construct a corresponding derived compound through a hierarchical proximal policy comprising a parent (molecular reaction) model 184 and a child (reactant) model 186 using an environment 154 of the target macromolecule, thereby generating a corresponding plurality of derived compounds. For instance, the experience illustrated in FIG. 5 begins with an initial compound 168-1-1 in state t=0 and culminates in a derived compound 180. An environment 154 of the target macromolecule is less than all of the target macromolecule. In some embodiments, an environment 154 of the target macromolecule is a small model (e.g., 20-400 atoms) of the most important residues cut from the active site (e.g., binding pocket) of the target macromolecule.

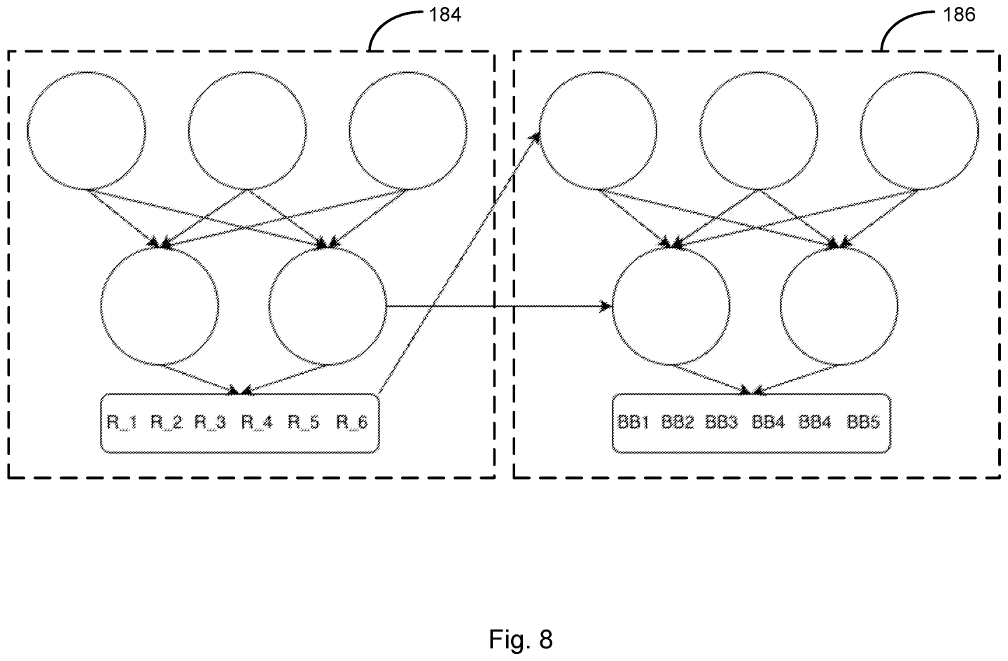

In accordance with block 624 of FIG. 3, described in further detail below in conjunction with the description of FIG. 6, the parent model 184 is a molecular reaction model that evaluates a plurality of molecular reactions (e.g., 212-1, 212-2, . . . , 212-P of molecular reaction data store 158 of FIG. 2A), while the child model 186 is a reactant model that evaluates a corresponding plurality of reactants (e.g., 214-1, 214-2, . . . , 214-T of reactant data store 160 of FIG. 2B) for a selected molecular reaction 212. An example hierarchical relationship between an example parent model 184 and child model 186 is illustrated in FIG. 8. As illustrated in FIG. 8, the output of parent model 184 is a probability for each of six molecular reactions, R_1, . . . , R_6. The probabilities for R_1, . . . , R_6 sum to one. One of the molecular reactions R_1, . . . , R_6 is selected (sampled) on a probabilistic basis. For example, if the parent model 184 assigned reaction R_1 a probability of 24%, there is a 24% chance that R_1 is selected. Next, the child model 186 takes the selected reaction and determines a probability for each reactant that could react with an initial compound in state t given the sampled molecular reaction. As illustrated in FIG. 8, the output of child model 186 is a probability for each of five reactants, BB_1, . . . , BB_5. The probabilities for BB_1, . . . , BB_5 sum to one. One of the reactants BB_1, . . . , BB_5 is selected (sampled) on a probabilistic basis. For example, if the child model 186 assigned reactant BB_3 a probability of 14%, there is a 14% chance that BB_3 is selected.

In accordance with block 630 of FIG. 3, described in further detail below in conjunction with the description of FIG. 6, the parent model 184 comprises a first plurality of parameters (e.g., 218-1, 218-2, . . . , 218-V, where V is a positive integer), and the child model 186 comprises a second plurality of parameters (e.g., 220-1, 220-2, . . . , 220-W, where V is a positive integer).

In accordance with block 686 of FIG. 3, described in further detail below in conjunction with the description of FIG. 6, the first plurality of parameters of the parent model 184 is updated in accordance with a first surrogate objective 188 calculated using the plurality of experiences 164-1, 164-2, . . . , 164-M.

In accordance with block 690 of FIG. 3, described in further detail below in conjunction with the description of FIG. 6, the second plurality of parameters of the child model 186 are updated in accordance with a second surrogate objective 190 using the plurality of experiences 164-1, 164-2, . . . , 164-M.

In accordance with block 690 of FIG. 3, described in further detail below in conjunction with the description of FIG. 6, blocks 612, 686, and 690 are repeated until a threshold convergence criterion is satisfied.

In accordance with block 694 of FIG. 3, described in further detail below in conjunction with the description of FIG. 6, a subset of the plurality of derived compounds 180, from the plurality of experiences, is tested in an assay (e.g., a wet lab assay) for activity against the target macromolecule, thereby identifying one or more derived compounds that exhibit the threshold activity with respect to the target macromolecule.

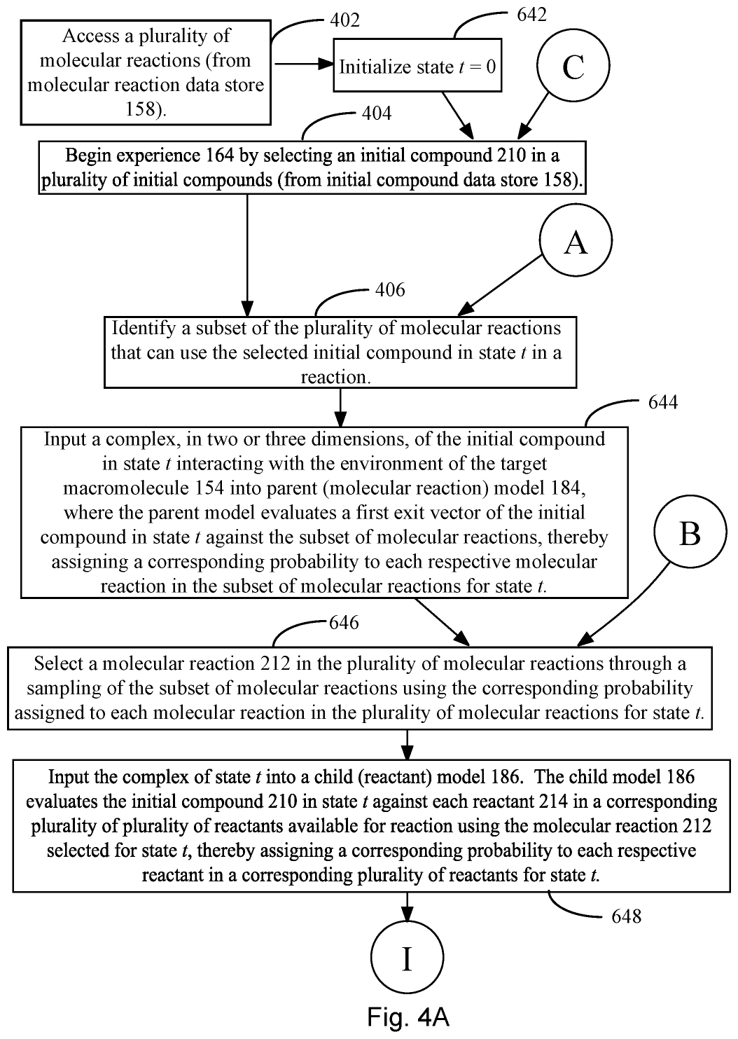

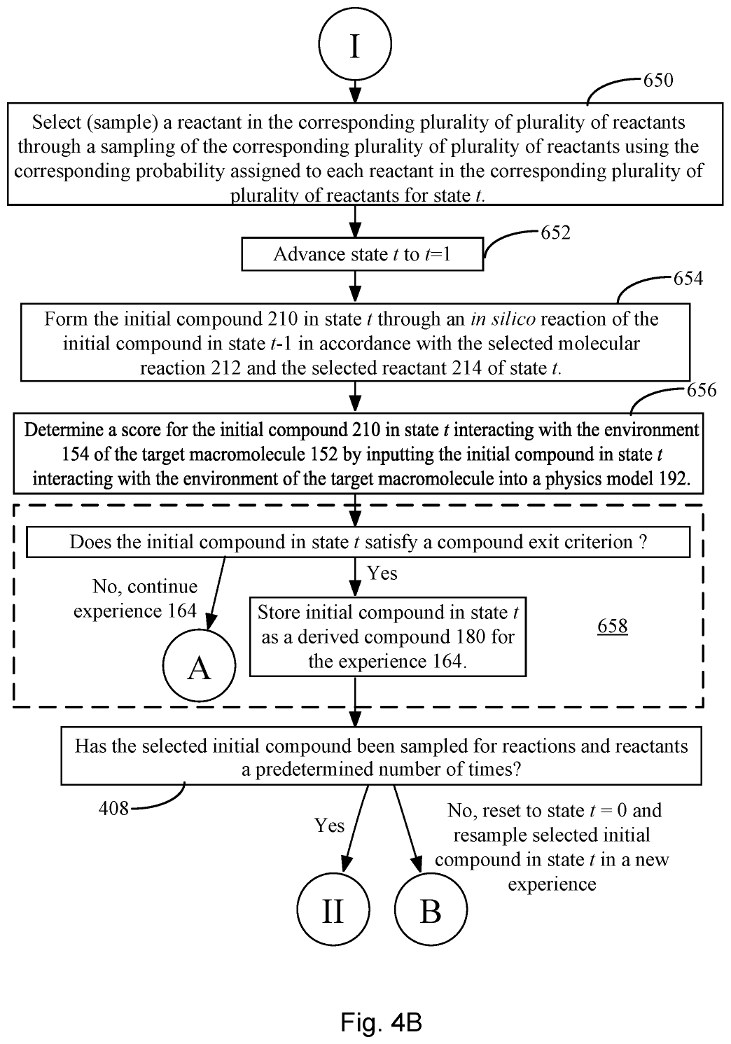

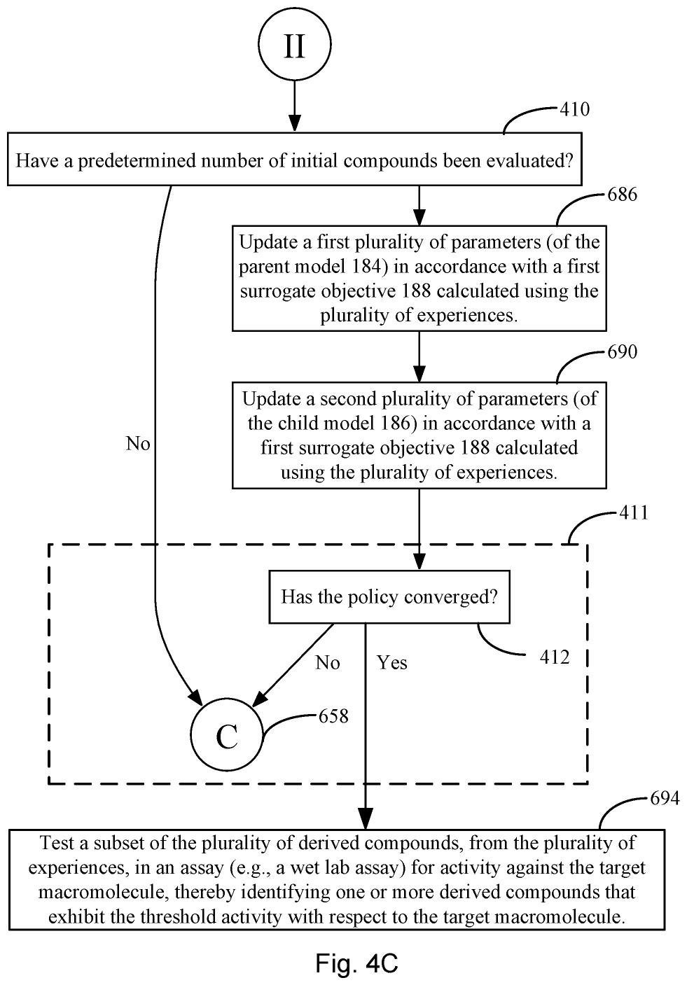

Now that an overview of a method for identifying one or more derived compounds that exhibit a threshold activity with respect to a target macromolecule has been described in conjunction with FIGS. 3 and 5, further details of methods for identifying such compounds is disclosed with reference to FIGS. 4 and 6.

Block 600. Referring to block 600 of FIG. 6A, a method is provided for identifying one or more derived compounds 180 that exhibit a threshold activity with respect to a target macromolecule 152, using a plurality of initial compounds. In some embodiments, as discussed above in conjunction with FIGS. 1, 2A, and 2B, the method is performed at a computer system 100 comprising one or more processing cores and a memory. In particular, in some embodiments of the present disclosure, the method is performed by a hierarchical reinforcement learning module 150 resident on, or electronically accessible by, computer system 100.

Block 602. Referring to block 602, in some embodiments, the target macromolecule 152 is a protein, a polypeptide, a polynucleic acid, a polyribonucleic acid, a polysaccharide, or an assembly of any combination thereof. In some embodiments, the target macromolecule 152 is a protein, a polypeptide, a polynucleic acid, a polyribonucleic acid, a polysaccharide, or an assembly of any combination thereof. In some embodiments, the target macromolecule 152 is a large molecule composed of repeating residues. In some embodiments, the target macromolecule 152 is a natural material. In some embodiments, the target macromolecule 152 is a synthetic material. In some embodiments, the target macromolecule 152 is an elastomer, shellac, amber, natural or synthetic rubber, cellulose, Bakelite, nylon, polystyrene, polyethylene, polypropylene, polyacrylonitrile, polyethylene glycol, or a polysaccharide.

In some embodiments, the target macromolecule 152 is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer comprises at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g., (A-B-A-B-B-A-A-A-A-B-B-B)ₙ). Additional examples of copolymers are statistical copolymers in which the sequence of monomer residues in the copolymer follows a statistical rule. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the target macromolecule 152 is a plurality of polymers (e.g., 2 or more, 3, or more, 10 or more, 100 or more, 1000 or more, or 5000 or more polymers), where the respective polymers in the plurality of polymers do not all have the same molecular weight. In some such embodiments, the polymers in the plurality of polymers share at least 50 percent, at least 60 percent, at least 70 percent, at least 80 percent, or at least 90 percent sequence identity and fall into a weight range with a corresponding distribution of chain lengths. In some embodiments, the target macromolecule 152 is a branched polymer molecule comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, the target macromolecule 152 is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine, as nonlimiting examples, are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

In some embodiments, the target macromolecule 152 includes any number of posttranslational modifications. Thus, in some embodiments, a target macromolecule 152 includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are within the scope of the macromolecules or macromolecule complexes of the present disclosure.

In some embodiments, the target macromolecule 152 is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecule contains both a water insoluble (or oil soluble) component and a water-soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water-soluble head group remains in the water phase. This alignment of surfactant molecules at the surface modifies the surface properties of water at the water/air or water/oil interface. Examples of ionic surfactants include ionic surfactants such as anionic, cationic, or zwitterionic (ampoteric) surfactants.

In some embodiments, the target macromolecule 152 is a reverse micelle or liposome. In some embodiments, the target macromolecule is a fullerene. A fullerene is any molecule composed entirely of carbon, in the form of a hollow sphere, ellipsoid or tube. Spherical fullerenes are also called buckyballs, and they resemble the balls used in association football. Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

In some embodiments, the target macromolecule 152 includes two different types of polymers, such as a nucleic acid bound to a polypeptide. In some embodiments, the target macromolecule includes two polypeptides bound to each other. In some embodiments, the target macromolecule 152 includes one or more metal ions (e.g., a metalloproteinase with one or more zinc atoms).

In some embodiments, the target macromolecule 152 comprises 50 or more, 100 or more, 150 or more, 200 or more, 300 or more, 400 or more, 500 or more, 600 or more, 700 or more, 800 or more, 900 or more, 1000 or more, or 5000 or more atoms. In some embodiments, the target macromolecule 152 comprises no more than 10,000, no more than 5000, no more than 1000, no more than 500, or no more than 100 atoms. In some embodiments, the target macromolecule 152 consists of from 50 to 100, from 50 to 500, from 100 to 1000, or from 1000 to 10,000 atoms. In some embodiments, the target macromolecule 152 comprises another range of atoms starting no lower than 50 atoms and ending no higher than 10,000 atoms.

In some embodiments, the target macromolecule 152 is a polymer comprising 10 or more, 20 or more, 30 or more, 50 or more, 100 or more, or 500 or more residues. In some embodiments, the target macromolecule 152 is a polymer comprising no more than 1000, no more than 500, no more than 100, no more than 50, or no more than 20 residues. In some embodiments, the target macromolecule 152 is a polymer consisting of from 10 to 100, from 50 to 200, from 100 to 500, or from 500 to 1000 residues. In some embodiments, the target macromolecule 152 is a polymer that falls within another range starting no lower than 10 residues and ending no higher than 1000 residues.

In some embodiments, the target macromolecule 152 comprises one or more active sites to which an initial compound and/or a derived compound can bind.

Block 604-606. Referring to block 604, in some embodiments, each initial compound 210 in the plurality of initial compounds is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Dalton, or less than 1000 Daltons. Referring to block 606, in some embodiments, each initial compound 210 in the plurality of initial compounds is an organic compound having a molecular weight of between 500 Daltons and 1000 Daltons.

In some embodiments, each initial compound 210 in state t=0 is an organic compound having a molecular weight of less than 50 Daltons, less than 100 Daltons, less than 150 Daltons, less than 200 Daltons, less than 250 Daltons, less than 300 Daltons, less than 400 Daltons, less than 500 Dalton, or less than 1000 Daltons. Referring to block 606, in some embodiments, each initial compound 210 in the plurality of initial compounds in state t=0 is an organic compound having a molecular weight of between 500 Daltons and 1000 Daltons.

In some embodiments, an initial compound has a molecular weight of at least 100, at least 500, at least 1000, at least 2000, at least 5000, or at least 10,000 Daltons. In some embodiments, an initial compound has a molecular weight of no more than 20,000, no more than 10,000, no more than 8000, no more than 6000, no more than 4000, no more than 2000, no more than 1000, or no more than 500 Daltons. In some embodiments, an initial compound has a molecular weight of from 100 to 500, from 500 to 2000, from 1000 to 8000, or from 5000 to 20,000 Daltons. In some embodiments, an initial compound has a molecular weight that falls within another range starting no lower than 100 Daltons and ending no higher than 20,000 Daltons. However, some embodiments of the disclosed systems and methods have no limitation on the size of an initial compound.

In some embodiments, each respective initial compound (e.g., in the plurality of initial compounds) is a chemical compound. In some embodiments, each respective initial compound (e.g., in the plurality of initial compounds) is a ligand. In some embodiments, a respective initial compound is an organic or inorganic compound.

In some embodiments initial compounds (e.g., initial compound in state t=0) are drawn from databases such as MCULE (Kiss et al., 2012, “Http://Mcule.Com: A Public Web Service for Drug Discovery,” J. Cheminformatics 4 (1), p. 17.) and ENAMINE (Irwin et al., 2016, “Docking Screens for Novel Ligands Conferring New Biology,” J. Med. Chem. 59 (9), pp. 4103-4120), each of which is hereby incorporated by reference.

Block 608. Referring to block 608, in some embodiments, each initial compound 210 in the plurality of initial compounds (e.g., initial compound in state 1=0) satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the initial compound satisfies one or more criteria in addition to Lipinski's Rule of Five. For example, in some embodiments, the initial compound has five or fewer aromatic rings, four or fewer aromatic rings, three or fewer aromatic rings, or two or fewer aromatic rings. In some embodiments, rather than imposing Lipinski's rule of Five requirements on the initial compounds, such requirements are imposed on the derived compounds as further detailed below in block 618. Rather, in some embodiments, user specified handcrafted physical constraints are imposed on the initial compound in state t=0, such as a molecular weight range (e.g., less than 400, less than 350, less than 300, less than 250 Daltons), log P range, maximum number of hydrogen bond donors/acceptors, maximum number or rotatable bonds, etc.

Block 610. Referring to block 610, in some embodiments, the plurality of initial compounds comprises 100 or more, 500 or more, 1000 or more, 2000 or more, 10,000 or more, 100,000 or more, 1×10⁶ or more, 1×10⁷ or more, or 1×10⁸ or more initial compounds.

Advantageously, the systems and methods of the present disclosure are designed to evaluate a large number of initial compounds. In some embodiments, the plurality of initial compounds comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ initial compounds. In some embodiments, the plurality of initial compounds comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 initial compounds. In some embodiments, the plurality of initial compounds consists of from 1000 to 100,000, from 10,000 to 1×10⁷, from 1×10⁶ to 1×10⁸, from 1×10⁸ to 1×10¹¹, or from 1×10⁹ to 1×10¹² initial compounds. In some embodiments, the plurality of initial compounds falls within another range starting no lower than 1000 candidate molecules and ending no higher than 1×10¹² initial compounds.

### Generate a Plurality of Experiences.

Block 612. Referring to block 612, a plurality of experiences is generated. Each respective experience 164 in the plurality of experiences uses an initial compound 210 selected from the plurality of initial compounds (e.g., of the initial compound data store 156) to construct a corresponding derived compound 180 through a hierarchical proximal policy 182 comprising a parent (molecular reaction) model 184 and a child (reactant) model 186 using an environment 154 of the target macromolecule, thereby generating a plurality of derived compounds. An example of an experience 164, beginning with an initial compound 168 in state t=0 through a final derived compound 180 is illustrated in FIG. 5.

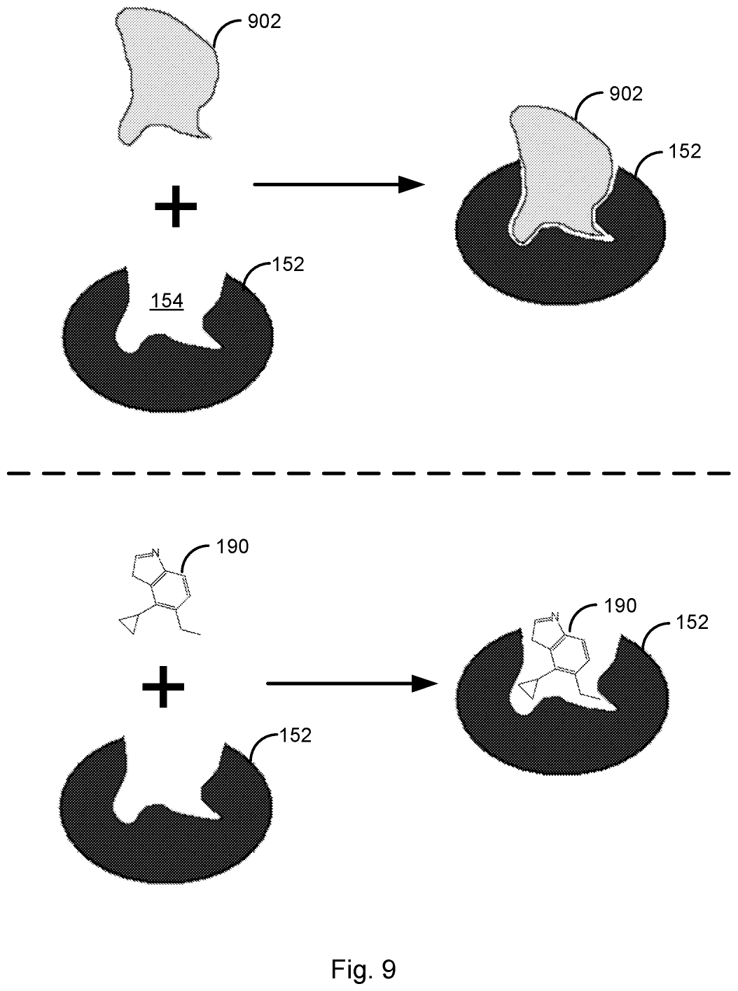

Block 614. Referring to block 614, in some embodiments, the environment of the target macromolecule 154 is a binding pocket of the target macromolecule 152. A stylized view of a target macromolecule 152 with an environment 154 that is a binding pocket is illustrated in FIG. 9, upper panel, in accordance with the prior art. Further illustrated in FIG. 9, upper panel is a natural ligand 902 for the target macromolecule 152, both before (FIG. 9, upper panel left), and after (FIG. 9, upper panel, right) forming a complex with the environment 154 (binding pocket) of the target macromolecule 152. The goal of an experience 164 is to derive a compound, such as compound 180 illustrated in to the lower panel of FIG. 9 that binds well to the environment of the target molecule.

In some embodiments, the environment of the target macromolecule 154 (e.g., a binding pocket) has a volume that ranges from 300 to 1,200 cubic angstroms (Å³). In some embodiments, the environment of the target macromolecule 154 has a volume that ranges from 250 to 5000 cubic Angstroms (Å³). In some embodiments, the environment of the target macromolecule 154 (e.g., a binding pocket) has a surface area that ranges between 400 and 1,200 square Angstroms (Å²).

Block 616. Referring to block 616, in some embodiments, the environment of the target macromolecule 154 is defined by a plurality of atomic coordinates of atoms of residues of the binding pocket derived by X-ray crystallography, neutron diffraction, cryo-electron microscopy, sampling from computational simulations, homology modeling, rotamer library sampling, or any combination thereof.

In some embodiments, the target macromolecule 152 is defined by a plurality of atomic coordinates {x₁, . . . , x_{N}} for a crystal structure of the target macromolecule 152, including the environment 154 of the target macromolecule, resolved at a resolution of 2.5 Å or better, where N is an integer of two or greater (e.g., 10 or greater, 20 or greater, etc.). In some embodiments, the target macromolecule 154 is a polymer and the spatial coordinates are a set of three-dimensional coordinates {x₁, . . . , x_{N}} for a crystal structure of the polymer resolved at a resolution of 3.3 Å or better. In some embodiments, the target macromolecule 152 is defined by a plurality of atomic coordinates {x₁, . . . , x_{N}} for a crystal structure of the macromolecule resolved (e.g., by X-ray crystallographic techniques) at a resolution of 3.3 Å or better, 3.2 Å or better, 3.1 Å or better, 3.0 Å or better, 2.5 Å or better, 2.2 Å or better, 2.0 Å or better, 1.9 Å or better, 1.85 Å or better, 1.80 Å or better, 1.75 Å or better, or 1.70 Å or better.

In some embodiments, the spatial coordinates of the target macromolecule 152 are an ensemble of ten or more, twenty or more or thirty or more three-dimensional coordinates for the target macromolecule 152 determined by nuclear magnetic resonance where the ensemble has a backbone RMSD of 1.0 Å or better, 0.9 Å or better, 0.8 Å or better, 0.7 Å or better, 0.6 Å or better, 0.5 Å or better, 0.4 Å or better, 0.3 Å or better, or 0.2 Å or better. In some embodiments the spatial coordinates of the target macromolecule 152 are determined by neutron diffraction or cryo-electron microscopy.

In some embodiments the spatial coordinates of the target macromolecule 152 are determined by a modeling program, such as AlphaFold2. AlphaFold2 is described in Jumper et al., 2021, “Highly accurate protein structure prediction with AlphaFold,” Nature 596, pp. 583-589; and Tunyasuvunakool et al., 2021, “Highly accurate protein structure prediction for the human protcome,” Nature 596, 590-596, each of which is hereby incorporated by reference.

Blocks 618-622. Referring to block 618, in some embodiments, each derived compound 180 in the plurality of derived compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons.

Referring to block 620, in some embodiments, each derived compound 180 in the plurality of derived compounds is an organic compound having a molecular weight of between 400 Daltons and 10000 Daltons.

Referring to block 622, in some embodiments, a derived compound 180 satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, a derived compound 180 satisfies one or more criteria in addition to Lipinski's Rule of Five. For example, in some embodiments, the derived compound has five or fewer aromatic rings, four or fewer aromatic rings, three or fewer aromatic rings, or two or fewer aromatic rings.

In some embodiments, a derived compound 180 satisfies Veber's rules: (i) the number of rotatable bonds (≤10) and the total polar surface area (TPSA) (≤140 Å²). In some embodiments, each derived compound 180 satisfies Veber's rules. See, Kralj et al., “Molecular Filters in Medicinal Chemistry,” Encyclopedia 2023, 3, 501-511, and Veber et al., 2002, “Molecular Properties That Influence the Oral Bioavailability of Drug Candidates,” J. Med. Chem. 45, 2615-2623, each of which is hereby incorporated by reference.

In some alternative embodiments, a derived compound 180 satisfies a Ghose filter: log P (octanol-water partition coefficient), molecular weight (160-480 Da), molar refractivity (40-130), and the number of atoms (20-70). In some embodiments, each derived compound 180 satisfies a Ghose filter. See, Kralj et al., “Molecular Filters in Medicinal Chemistry,” Encyclopedia 2023, 3, 501-511, and Ghose et al., 1999, “A Knowledge-Based Approach in Designing Combinatorial or Medicinal Chemistry Libraries for Drug Discovery, 1. A Qualitative and Quantitative Characterization of Known Drug Databases,” J. Comb. Chem. 1, pp. 55-68, each of which is hereby incorporated by reference.

In some embodiments, a derived compound 180 satisfies Egan's filter: compound has a log P≤5.88 and a total polar surface area of ≤131.6 Å². In some embodiments, each derived compound 180 satisfies Egan's filter. See, Egan et al., 2000 “Prediction of Drug Absorption Using Multivariate Statistics,” J. Med. Chem. 43, pp. 3867-3877 each of which is hereby incorporated by reference.

In some embodiments, a derived compound 180 satisfies Muegge's rule: molecular weight (200-600 Daltons), log P (−2 to 5), PSA≤150, number of rings (≤7), and number of rotatable bonds (≤15), number of carbons >4, number of heteroatoms >1, number of hydrogen bond donors≤5. In some alternative embodiments, each derived compound satisfies Muegge's rule. See, Vélez et al, 2022, “Theoretical calculations and analysis method of the physicochemical properties of phytochemicals to predict gastrointestinal absorption,” Int. J. Plant Biol. 13(2), pp. 163-179, which is hereby incorporated by reference.

Block 624. Referring to block 624, in some embodiments, the parent model 180 is a molecular reaction model that evaluates a plurality of molecular reactions, and the child model is a reactant model that evaluates a corresponding plurality of reactants for a molecular reaction. An example of such a parent/child relationship between an example parent model 184 and child model 186 is illustrated in FIG. 8. As illustrated in FIG. 8, the output of parent model 184 is a probability for each of six molecular reactions, R_1, . . . , R_6. One of the molecular reactions R_1, . . . , R_6 is selected (sampled) on a probabilistic basis. For example, if the parent model 184 assigned reaction R_1 a probability of 24%, there is a 24% chance that R_1 is selected. Next, the child model 186 takes the selected reaction and determines a probability for each reactant that could react with an initial compound in state t given the sampled molecular reaction. As illustrated in FIG. 8, the output of child model 186 is a probability for each of five reactants, BB_1, . . . , BB_5, one of which is selected (sampled) on a probabilistic basis. For example, if the child model 186 assigned reactant BB_3 a probability of 14%, there is a 14% chance that BB_3 is selected.

Block 626. Referring to block 626, in some embodiments, the parent model 180 is a first graph neural network (e.g., a first graph isomorphism neural network). Graph isomorphism networks are disclosed in Hu et al., 2018, “How Powerful are Graph Neural Networks,” cs>arXiv:1810.00826, which is hereby incorporated by reference.

In some embodiments, the parent model 180 is deep graph convolutional neural network (e.g., Zhang et al, “An End-to-End Deep Learning Architecture for Graph Classification,” The Thirty-Second AAAI Conference on Artificial Intelligence), GraphSage (e.g., Hamilton et al., 2017, “Inductive Representation Learning on Large Graphs,” arXiv:1706.02216 [cs.SI]), a graph isomorphism network (e.g., Hu et al., 2018, “How Powerful are Graph Neural Networks,” cs>arXiv:1810.00826, an edge-conditioned convolutional neural network (ECC) (e.g., Simonovsky and Komodakis, 2017, “Dynamic Edge-Conditioned Filters in Convolutional Neural Networks on Graphs,” arXiv:1704.02901 [cs.CV]), a differentiable graph encoder such as DiffPool (e.g., Ying et al., 2018, “Hierarchical Graph Representation Learning with Differentiable Pooling” arXiv:1806.08804 [cs.LG]), a message-passing graph neural network such as MPNN (Gilmer et al., 2017, “Neural Message Passing for Quantum Chemistry,” arXiv:1704.01212 [cs.LG]) or D-MPNN (Yang et al., 2019, “Analyzing Learned Molecular Representations for Property Prediction” J. Chem. Inf. Model. 59(8), pp. 3370-3388), or a graph neural network such as CMPNN (Song et al., “Communicative Representation Learning on Attributed Molecular Graphs,” Proceedings of the Twenty-Ninth International Joint Conference on Artificial Intelligence (IJCAI-20)). See also Rao et al., 2021, “MolRep: A Deep Representation Learning Library for Molecular Property Prediction,” doi.org/10.1101/2021.01.13.426489; posted Jan. 16, 2021. T; Rao et al., “Quantitative Evaluation of Explainable Graph Neural Networks for Molecular Property Prediction,” arXiv preprint arXiv:2107.04119; and github.com/biomed-AI/MolRep, for additional models that can be used as the parent model. In some embodiments, the parent model 180 has any of the architectures disclosed herein.

Referring to block 628, in some embodiments, the child model 186 is a second graph neural network (e.g., a second graph isomorphism neural network) that is passed an output of the parent model. In some embodiments, the architecture of the child model is the same or different than the architecture of the parent model and can have any of the architectures described in block 626.

Block 630. Referring to block 630, in some embodiments, the parent model 184 comprises a first plurality of parameters 218-1, 218-2, . . . , 218-V, where V is a positive integer (e.g., at least 10,000, at least 100,000, or at least 1×10⁶ parameters), and the child model 186 comprises a second plurality of parameters 220-1, 220-2, . . . , 220-W, where W is a positive integer (e.g., at least 10,000, at least 100,000, or at least 1×10⁶ parameters).

In some embodiments, the first plurality of parameters 218 comprises at least 10, at least 100, at least 1000, at least 10,000, at least 100,000, at least 1×10⁶, at least 1×10⁷, or more parameters. In some embodiments, the first plurality of parameters consists of no more than 1×10⁸, no more than 1×10⁷, no more than 1×10⁶, no more than 100,000, no more than 10,000, no more than 1000, or no more than 100 parameters. In some embodiments, the first plurality of parameters consists of from 10 to 1000, from 100 to 100,000, from 10,000 to 1×10⁷, or from 1×10⁶ to 1×10⁸ parameters. In some embodiments, the first plurality of parameters falls within another range starting no lower than 10 parameters and ending no higher than 1×10⁸ parameters.

In some embodiments, the second plurality of parameters 220 comprises at least 10, at least 100, at least 1000, at least 10,000, at least 100,000, at least 1×10⁶, at least 1×10⁷, or more parameters. In some embodiments, the second plurality of parameters consists of no more than 1×10⁸, no more than 1×10⁷, no more than 1×10⁶, no more than 100,000, no more than 10,000, no more than 1000, or no more than 100 parameters. In some embodiments, the second plurality of parameters consists of from 10 to 1000, from 100 to 100,000, from 10,000 to 1×10⁷, or from 1×10⁶ to 1×10⁸ parameters. In some embodiments, the second plurality of parameters falls within another range starting no lower than 10 parameters and ending no higher than 1×10⁸ parameters.

Block 632. Referring to block 632, in some embodiments, the plurality of molecular reactions comprises named reactions, organic synthesis reactions or protecting group reactions.

In some embodiments, the plurality of molecular reactions comprises at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 10 molecular reactions and ending no higher than 5000 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, a molecular reaction in the plurality of molecular reactions is represented by a Simplified Molecular Input Line Entry Specification (SMILES) arbitrary target specification ((SMARTS). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups, total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, methyl esterification, hydrolysis of esters, amide synthesis, transamidation, oxidative amidation, Schmidt Reaction, Schotten-Baumann Reaction, Ugi Reaction, arylamine synthesis, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki Coupling, Sonogashira Coupling, Click Chemistry, Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, Synthesis of 1,2,3-Triazoles, epoxide synthesis, Jacobsen-Katsuki Epoxidation, Prilezhacv Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

Blocks 634-638. Referring to block 634, in some embodiments, the corresponding plurality of reactants is a corresponding plurality of synthons. Referring to block 636, in some embodiments, the corresponding plurality of reactants comprises twenty or more reactants. Thus, in such embodiments, the child model evaluates and assigns a probability to each of twenty or more reactants, where the probabilities sum to one. For example, referring to state t=1 of FIG. 5 where a substitution reaction is selected, in instances where the corresponding plurality of reactants consists of twenty reactants, twenty different substitution groups (reactants) are evaluated for substituting out the bromide atom from the initial compound in state 1, and the child model assigns each of these substitution groups a probability, where the collective probabilities assigned to the twenty different substitution groups by the child model sum to one. The twenty different substitution groups are then sampled based on the assigned probabilities to select the actual substation that will be used in the chemical reaction selected in state 1 in order to build the initiation compound in state 2.

Referring to block 638, in some embodiments, the corresponding plurality of reactants comprises 20 or more synthons, 50 or more synthons, 100 or more synthons, 1000 or more synthons, 10,000 or more synthons, 100,000 or more synthons, or 1×10⁶ or more synthons. As used herein, a “synthon” refers to a representation of a chemical structure having an open valence (attachment bond) at, at least, one position. In some embodiments, synthons are derived from a reagent, from a synthetic reaction sequence, or from the fragmentation of a molecule (e.g., chemical structures derived from the disconnection of a bond). The potential universe of synthons can be vast. Synthons are building blocks or molecular fragments that can be combined in different ways to produce a wide range of compounds. In some embodiments the pool of possible synthons (e.g., in initial compound data store 158) considered represents more than 100, 500, 1000, 2000, 5000, 10,000, 11,000, 12,000, 13,000, 14,000, 15,000, or 20,000 synthons. In some embodiments these synthons might include various functional groups, heterocycles, and other structural motifs. In some embodiments, however, only those synthons, from this universe of synthons, that can work in the molecular reaction identified by the parent model, against a vector (reactive group) of the subject initial compound are considered by the child model during any given state of a particular experience.

Block 640. Referring to block 640 of FIG. 6D, in some embodiments, an experience 164 in the plurality of experiences is generated by the procedure outlined by blocks 640 through 658 in FIGS. 6D-6F, described in further detail below, and as further illustrated in FIGS. 4A, 4B, and 4C. At the outset, as illustrated in element 402 of FIG. 4A, a plurality of molecular reactions is accessed. A description of suitable molecular reactions that can be accessed is described above in conjunction with block 632.

Block 642. At block 642 (i) the experience 164 is initialized to state t=0, as illustrated in FIG. 4A. Referring to element 404 of FIG. 4A, state t=0 represents the selection of an initial compound before any in silico molecular reaction has been performed on the initial compound. In some embodiments, an initial compound at state t=0 is a compound randomly obtained from a chemical diversity library such as ENAMINE REAL. See, Shivanyuk et al., “Enamine real database: Making chemical diversity real,” Chem Today [Internet], 2007 [cited 2024 Apr. 11], Available from: https://elibrary.ru/item.asp?id=27792199, which is hereby incorporated by reference.

Referring to block 406 of FIG. 4A, in some embodiments, once an initial compound has been selected, the plurality of molecular reactions is filtered to identify a subset of molecular reactions that can make use of the selected molecular reaction. For example, referring to state t=0 in FIG. 5, one molecular reaction that can make use of the initial compound in state 0 is a halogenation reaction. Accordingly, a halogenation reaction is one of the molecular reactions that is included in the subset of molecular reactions in accordance with block 406 in some embodiments.

Block 644. At block 644 (ii) a complex, in two or three dimensions, of the initial compound 210 in state t interacting with the environment 154 of the target macromolecule 152 is inputted into the parent model 184. In some embodiments, to perform block 644, the initial compound 210 in state t is first docked into the environment (e.g., binding pocket) of the target macromolecule. A nonlimiting example of such docking programs is described above in conjunction with the definition of “pose” in the definitions section. The three dimensional coordinates of the complex of the compound 210 in state t with the environment (e.g., binding pocket) of the target macromolecule is then inputted into a parent model in some embodiments. In alternative embodiments, the three dimensional coordinates of the complex of the compound 210 in state t with the environment (e.g., binding pocket) of the target macromolecule is first converted into a two-dimensional graph and then inputted into the parent model. Example programs and techniques for generating a two-dimensional graph of a three dimensional complex are disclosed in Xu et al., “How powerful are graph neural networks?” ICLR 2019, arXiv:1810.00826v3, which is hereby incorporated herein by reference in its entirety. In such embodiments, the nodes of the graph typically represent atoms and the edges between the nodes represent bonds or interactions (e.g., covalent bonds, hydrogen bonds, or van der Waals interactions) between the atoms of the complex. In some such embodiments, the three-dimensional coordinates of the atoms of the initial compound complexed with the environment of the target macromolecule, and the information about their chemical environment (such as atom types, bond types, etc.) is fed into a model such as a graph neural network. The model encode the spatial relationships and interactions from the three dimensional complex into a lower-dimensional representation. After processing the three-dimensional complex, the model can output a two-dimensional graph where the spatial information is implicitly captured in the node and edge features. This two-dimensional graph can, in turn, be evaluated by the parent model. The parent model 184 evaluates a first exit vector of the initial compound in state t against the plurality of molecular reactions, thereby assigning a corresponding probability to each respective molecular reaction in the molecular reactions considered for state t. For instance, in FIG. 5, the bromine of the initial compound in state 1 is the exit vector considered in state 1 of the experience illustrated in FIG. 5. In some embodiments, the parent model evaluates and provides a probability for 2, 3, 4, 5, 6, 7, 8, 9, or 10 different molecular reactions. In some embodiments, the parent model evaluates and provides a probability for 10, 20, 30, 40, 50, 60, 70, 80, 90, or 100 or more different molecular reactions. In such embodiments these probabilities sum to one.

Block 646. Referring to block 646, (iii) a molecular reaction 212 in the plurality of molecular reactions is selected, through a sampling of the plurality of molecular reactions using the corresponding probability assigned to each molecular reaction 212 in the plurality of molecular reactions for state t. For instance, in the example illustrated in FIG. 8, the output of parent model 184 is a probability for each of six molecular reactions, R_1, . . . , R_6. The probabilities assigned by the parent model for R_1, . . . , R_6 sum to one. One of the molecular reactions R_1, . . . , R_6 is selected (sampled) on a probabilistic basis. For example, if the parent model 184 assigned reaction R_1 a probability of 24%, there is a 24% chance that R_1 is selected in block 646 of FIG. 4A.

In some embodiments, block 646 is performed a number of times. Each time, a molecular reaction 212 in the plurality of molecular reactions is selected, through a sampling of the plurality of molecular reactions using the corresponding probability assigned to each molecular reaction 212 in the plurality of molecular reactions for state t. Each such sampling represents a different experience. In other words, referring to FIG. 4A, block 646 represents a branching to numerous different instances of block 648 and subsequent blocks, one for each instance of block 646 in such embodiments.

Blocks 648-650. Referring to block 648 (iv), the complex of state t is inputted into the child model 186.

In some embodiments, the complex of state t (the initial compound in state t docked into the environment of the target macromolecule) is in two or three dimensions in the same manner described for the input of the parent model in block 644 above.

The child model 186 evaluates the initial compound 210 in state t against each reactant 214 in a corresponding plurality of reactants available for reaction using the molecular reaction 212 selected for state t, thereby assigning a corresponding probability to each respective reactant in the corresponding plurality of reactants for state t. For example, as illustrated in FIG. 8, the child model 186 takes the selected molecular reaction of the parent model and the initial compound in state t (optionally complexed with the environment of the target macromolecule) and determines a probability for each reactant that could react with the initial compound in state t given this sampled molecular reaction.

Referring to block 650 of FIG. 6E, (v) a reactant 214 in the corresponding of plurality of reactants is selected through a sampling of the corresponding plurality of reactants using the corresponding probability assigned to each reactant in the corresponding plurality of reactants for state t. For instance, in the example illustrated in FIG. 8, the output of child model 186 is a probability for each of five reactants, BB_1, . . . , BB_5. The probabilities for BB_1, . . . , BB_5 sum to one. In accordance with block 650 of FIG. 6E, one of the reactants BB_1, . . . , BB_5 is selected (sampled) on a probabilistic basis. For example, if the child model 186 assigned reactant BB_3 a probability of 14%, there is a 14% chance that BB_3 is selected in block 650. As discussed above in conjunction with blocks 632 through 363, the actual number of reactants considered by the child model 186 can be a number other than five.

In some embodiments, block 650 is performed a number of times. Each time, a reactant 214 in the corresponding of plurality of reactants is selected through a sampling of the corresponding plurality of reactants using the corresponding probability assigned to each reactant in the corresponding plurality of reactants for state t. Each such sampling would represents a different experience. In other words, referring to FIG. 4B, block 650 represents a branching to numerous different instances of block 652 and subsequent blocks, one or each instance of block 640 in such embodiments.

Block 652. In block 652, (vi) the state is advanced from state t to state t+1 since a new molecule is about to be generated based on the initial compound at prior state t, the selected molecular reaction from block 646, and the selected reactant from block 650. In embodiments where the initial compound at prior state t has more than one vector (reactive atom or group), all other vectors are either removed from the initial compound at prior state t or are otherwise disregarded by the in silico synthesis.

Block 654. In block 654, (vii) the initial compound 210 in state t is formed through an in silico reaction of the initial compound in state t−1 in accordance with the selected molecular reaction 212 and the selected reactant 214 of state t. In some embodiments, a program such as Molgen version 3.5, 4, or 5, Molgen-COMB, or MOLGEN-QSPR is used to perform this in silico reaction. See, for example, the Molgen Reference Guide, Version 5.0, Mar. 9, 2021, available on the Internet at https://molgen.de/documents/manual_molgen50.pdf; Gugisch et al., 2000, “MOLGENCOMB, a Software Package for Combinatorial Chemistry,” Commun. Math. Comput. Chem. 41 pp. 189-203; and Kerber et al., “MOLGEN-QSPR, a software package for the study of quantitative structure property relationships,” MATCH—Communications in Mathematical and in Computer Chemistry 51, each of which is hereby incorporated by reference. In some embodiments, alternatives to Molgen, such as RDKit, ChemAxon's Reactor, and Schrödinger's Maestro and Reaction-based Tools is used in block 654. See, for example Saldívar-González et al., 2020, “Chemoinformatics-based enumeration of chemical libraries: a tutorial,” J Cheminform (2020) 12:64; and Landrum, 2020, “RDKit,” https://www.rdkit.org/, Accessed Aug. 29, 2024, each of which is hereby incorporated by reference.

In some embodiments, block 654 produces numerous initial compounds in state t. In some embodiments, each such initial compounds in new state t has its own branch beginning with block 656 and subsequent blocs. Thus, each initial compound in new state t is scored in accordance with block 656 and evaluated by block 658. In some embodiments, block 654 produces numerous initial compounds in state t and assigns each such compound a probability. In such embodiments, these probabilities are sample to select a number of initial compounds in state t, each of which is evaluated in accordance with blocks 656 and 658 (and thus form their own experience).

Block 656. In block 656, (viii) a score for the initial compound 210 in state t interacting with the environment 154 of the target macromolecule 152 is determined by inputting the initial compound in state t interacting with the environment of the target macromolecule into a physics model 192.

In some embodiments, the score for the initial compound 210 in state t interacting with the environment 154 of the target macromolecule 152 characterizes or otherwise indicates an interaction between the initial compound 210 and the environment 154 of the target macromolecule 152. In some implementations, the score is a causal interaction feature score that is obtained using one or more interaction features associated with a conformation of the initial compound 210 in state t when complexed to the target macromolecule 152. However, in other embodiments, the score for the initial compound 210 in state t interacting with the environment 154 of the target macromolecule 152 is an interaction score obtained by other methods, as will be apparent to one skilled in the art.

In some embodiments, the score for the initial compound 210 in state t interacting with the environment 154 of the target macromolecule 152 is based at least on a count of interaction features for a conformation of the initial compound 210 in state t when complexed to the target macromolecule 152. A count of interaction features can refer to a tally of a plurality of interaction features associated with the initial compound 210 in state t, but can also refer to any weighted count or computation of causality over the plurality of interaction features considered by the physics model.

Further examples of interaction features that can be used by the physics model are described in block 674.

Accordingly, in some embodiments, the score for the initial compound 210 in state t interacting with the environment 154 of the target macromolecule 152 is an absolute count, a weighted count, an individual treatment score (e.g., a dot product between an interaction feature vector and corresponding average treatment effects for each respective interaction feature in an interaction feature vector), a weighted individual treatment score, an efficiency score (e.g., a ratio of the number of interaction features for the respective molecule and the number of heavy atoms in the respective molecule), a weighted efficiency score, a diversity score (e.g., a measure of a diversity of interaction feature classes in a plurality of interaction features associated with the initial compound 210 in state t interacting with the environment 154 of the target macromolecule 152), and/or a weighted diversity score.

In some implementations, a weighted score gives greater import to one or more interaction features in a corresponding plurality of interaction features for the initial compound 210 in state t, compared to other interaction features in the corresponding plurality of interaction features. In an example implementation, a weighted score gives greater weight to a first interaction feature that is selected as or known to be highly causal or associated with a particular property relevant to interaction (e.g., binding potency, selectivity, ADME properties, toxicity, etc.). In such an example implementation, the weighted score gives less weight to a second interaction feature that is selected as or known to be a covariate, confounder, or otherwise have lower causality for the particular property.

In some embodiments, the score is based, at least in part, on a calculated absorption, distribution, metabolism, and excretion (ADME) score. In some embodiments, an ADME model accepts, as input, a molecular fingerprint and/or a two-dimensional molecular graph of the initial compound in state t. Typically, drug development involves assessment of absorption, distribution, metabolism, and excretion (ADME) and/or toxicity (ADMET) to determine the effectiveness of an initial compound in state t as a drug. Such effectiveness is measured, in some implementations, as the ability of an initial compound in state t to reach its target in the subject in sufficient concentration, maintain bioactivity for long enough to achieve a target effect, and cause minimal toxicity. In some implementations, ADME or ADMET properties are determined using any one or more of a variety of techniques, including but not limited to substructure searches, molecular fingerprint methods, support vector machine (SVM) or Bayesian techniques, and/or deep neural networks. Various tools for predicting ADME or ADMET properties from the chemical structure of compounds are known in the art and provide indications of an initial compound in state t's physicochemical properties, pharmacokinetics, drug-likeness and/or medicinal chemistry friendliness, among others. Examples of such models include, but are not limited to, SwissADME, pk-CSN, admetSAR, iLOGP, BOILED-Egg, and/or Bioavailability Radar, each of which can be, or can contribute to the score of block 656.

Any number of ADME or ADMET models are contemplated for use in the present disclosure. For instance, available tools for predicting ADME or ADMET properties include those that focus on all or less than all ADME or ADMET properties. Accordingly, in some implementations, a plurality of ADME or ADMET models are used to determine a broad range of target properties, where each respective ADME or ADMET model outputs a corresponding measure of activity for the initial compound in state t that corresponds to one or more respective ADME or ADMET properties in a plurality of ADME or ADMET properties. ADME and ADMET models are further described, for example, in Daina et al., “SwissADME: a free web tool to evaluate pharmacokinetics, drug-likeness and medicinal chemistry friendliness of small molecules,” Sci Rep. 2017; 7(1):42717, which is hereby incorporated by reference in its entirety.

In some embodiments, the measure of activity determined to compute the score of block 656 includes a corresponding at least 1, at least 2, at least 3, at least 5, at least 10, or at least 20 measures of activity. In some embodiments, the corresponding measure of activity includes no more than 20, no more than 15, no more than 10, or no more than 5 measures of activity. In some embodiments, the corresponding measure of activity consists of from 1 to 5, from 2 to 10, from 5 to 18, or from 10 to 20 measures of activity. In some embodiments, the corresponding measure of activity falls within another range starting no lower than 1 and ending no higher than 20 measures of activity.

In some embodiments, a weighted score is differentially weighted based on the presence or absence of one or more interaction features in a corresponding plurality of interaction features for the initial compound 210 in state t. For instance, in some such embodiments, a respective score for the initial compound 210 in state t is predictive of binding when one or more interaction features, or classes thereof, in a first subset of interaction features is present in the corresponding plurality of interaction features for the initial compound 210 in state t, and is not predictive of binding when none of the interaction features, or classes thereof, in the first subset of interaction features is present in the corresponding plurality of interaction features for the initial compound 210 in state t. In other words, in some such embodiments, a weighted score accounts for interaction features or feature classes that are selected as or known to be essential for a particular interaction property. Alternatively or additionally, in some embodiments, a weighted score accounts for interaction features or feature classes that are selected as or known to be adverse or inhibitive to the particular interaction property. In some embodiments, a weighted score is determined by adjusting a corresponding attribute for each respective interaction feature by a weighting factor (e.g., 0.8, 0.2).

In some embodiments, interaction feature classes include any of the feature classes disclosed elsewhere herein, including but not limited to partial charge, H-bond acceptor, H-bond donor, aromatic ring, hydrophobic interaction, and/or other pharmacophores.

In some embodiments, a score for the initial compound 210 in state t interacting with the environment 154 of the target macromolecule 152 is obtained using a respective plurality of interaction features obtained for a complex formed between the initial compound 210 in state t interacting with the environment 154 of the target macromolecule 152.

One skilled in the art will appreciate that the interaction features used for calculating the score for the initial compound 210 in state t interacting with the environment 154 of the target macromolecule 152 can be obtained using any suitable method, including but not limited to a causal binding hypothesis generation method, a causal selectivity hypothesis generation method, a graph neural network for binding, and/or a graph neural network for selectivity.

In some embodiments, the score for the initial compound 210 in state t interacting with the environment 154 of the target macromolecule is in fact a composite score formed from individual component scores. For example, FIG. 2B illustrates physics model 192-1 and physics model 192-2. In some embodiments the score for the initial compound 210 in state t interacting with the environment 154 of the target macromolecule is determined by inputting the initial compound in state t interacting with the environment of the target macromolecule into both physics model 192-1 and physics model 192-2 with each model producing a component score that is aggregated to form the score for the initial compound 210 in state t interacting with the environment 154 of the target macromolecule. In some embodiments, there are 3, 4, 5, 6, 7, 8, 9, 10, 11, 12, 13, 14, or 15 or more physis models that each contribute a component score that is aggregated to form the score for the initial compound 210 in state t interacting with the environment 154 of the target macromolecule upon input of the initial compound 210 in state t interacting with the environment 154 of the target macromolecule.

In some embodiments, the score for the initial compound 210 in state t interacting with the environment 154 of the target macromolecule takes input (e.g., component score) from both one or more physics models as well as other kinds of models.

For instance, in a first example, in some embodiments the two-dimensional structure of the initial compound in state t is used to ensure that the compound is within the ideal cheminformatics ranges such as a user specified log p range, a user specified molecular weight range, is user specified range of hydrogen acceptors, a user specified quantitative estimate of drug-likeness (QED) score, a scaffold diversity measure, etc. In some embodiments, one or more component scores from such cheminformatic checks contributes to the score of block 656.

In some embodiments reactive handles (vectors) on the initial compound in state t are replaced with carbons to ensure that that reactive handles are being classified as making interactions with the environment 154 of the target macromolecule. The initial compound in state t is then docked to the environment 154 of the target macromolecule. In some such embodiments a docking score for this docking contributes to the score of block 656.

In some embodiments, the docking identifies multiple poses of the initial compound in state t docked to the environment 154 of the target macromolecule, each of which is scored, and each of which contributes to the score of block 656. In some embodiments, the best 5, 6, 7, 8, 9, 10, 15, 20, 25, 30, or 50 poses are taken and each contributes to the score of block 656.

In some embodiments, the single best pose or the top N poses, where N is a positive integer between 2 and 100, of the initial compound in state t docked to the environment 154 of the target macromolecule are evaluated for interaction hits. In some embodiments, the interactions that are evaluated are specified in a causal interaction feature contract for the environment 154 of the target macromolecule. Methods for identifying causal interaction features that can populate a causal interaction feature contract are disclosed in International Patent Application No. PCT/US24/24456, entitled “Systems and Methods for Discovering Compounds Using Causal Inference,” filed Apr. 12, 2024, which is hereby incorporated by reference. In some embodiments, one or more score for such interactions (e.g., one for each pose, or a composite of the poses) contributes to the score of block 656.

In some embodiments, the interaction energies between the single best pose or the top N poses and the environment 154 of the target macromolecule are evaluated using quantum mechanical calculations. One example suitable program for this is disclosed in Gao et al., “TorchANI: A Free and Open Source PyTorch-Based Deep Learning Implementation of the ANI Neural Network Potentials,” ChemRxiv. 2020; doi:10.26434/chemrxiv.12218294.v1, which is hereby incorporated by reference. In some embodiments, one or more score for such interactions (e.g., one for each pose, or a composite of the poses) contributes to the score of block 656.

In some embodiments, non-covalent interactions between the single best pose or the top N poses of the initial compound in state t docked to the environment 154 of the target macromolecule are evaluated using a symmetry-adapted perturbation theory (SAPT) zeroth-order approximation framework, which considers, for example, electrostatic interactions, exchange-repulsion interactions, induction, and dispersion of such complexes. One example suitable program for this is disclosed in Patkowski, 2019 “Recent developments in symmetry-adapted perturbation theory,” WIREs Computational Molecular Science 10(3), which is hereby incorporated by reference. In some embodiments, one or more score from such calculations (e.g., one for each pose, or a composite of the poses) contributes to the score of block 656.

In some embodiments, any combination of such scores is accumulated (aggregated) and used as the overall score computed in block 656. In some embodiments, the overall score is a measure of central tendency (e.g., mean, median, mode, weighted mean, weighted median, and/or weighted mode) of the component scores produced by any combination of the score techniques of the present disclosure.

In some embodiments a two-dimensional molecular graph of the initial compound in state t docked to the environment 154 of the target macromolecule is inputted into a model, and responsive to this input, the model provides, as output, a corresponding plurality of interaction features for the complex the initial compound in state t docked to the environment 154 of the target macromolecule as disclosed in International Patent Application No. PCT/US24/24456, entitled “Systems and Methods for Discovering Compounds Using Causal Inference,” filed Apr. 12, 2024, which is hereby incorporated by reference. The interaction features identified by the model can be used, at least in part, to determine a score for the initial compound in state t that is evaluated against the compound exit criterion of block 658. In some embodiments, such a model is a graph neural network model, a neural network (e.g., a multi-layer perceptron, a fully connected neural network, a partially connected neural network, etc.), a support vector machine, a Naive Bayes algorithm, a nearest neighbor algorithm, a boosted trees algorithm (e.g., XGBoost, LightGBM), a random forest algorithm, a decision tree algorithm, a logistic regression algorithm, a linear model, a linear regression algorithm, and/or any combination thereof. Various other model architectures are possible for use in obtaining, for an initial compound in state t docked to the environment 154 of the target macromolecule, a corresponding plurality of interaction features for the complex formed between the initial compound in state t docked to the environment 154 of the target macromolecule, as will be apparent to one skilled in the art. In some such embodiments, the model is trained as disclosed in International Patent Application No. PCT/US24/24456, entitled “Systems and Methods for Discovering Compounds Using Causal Inference,” filed Apr. 12, 2024, which is hereby incorporated by reference.

Alternatively or additionally, when the score comprises an individual treatment score calculated as a dot product of an interaction feature vector and corresponding average treatment effects (ATEs) of the respective interaction features as disclosed in International Patent Application No. PCT/US24/24456, entitled “Systems and Methods for Discovering Compounds Using Causal Inference,” filed Apr. 12, 2024, which is hereby incorporated by reference, the initial compound in state t fails to satisfy the criterion when the individual treatment score is greater than a threshold value (e.g., greater than −1, greater than −0.5, greater than −0.1, greater than 0, etc.). In general, because the individual treatment score is calculated using the ATEs of individual interaction features, and because ATEs are representative of the Gibbs free energy of a particular conformation of the initial compound in state t interacting with the environment 154 of the target macromolecule 152, higher individual treatment scores are predictive of poor overall binding affinity or specificity.

Block 658. In accordance with block 658, (ix) elements (ii), (iii), (iv), (v), (vi), (vii), and (viii) are repeated until a compound exit criterion (e.g., the compound exit criterion comprises a molecular weight, a molecular weight range, a log p, or a log p range) is satisfied by the initial compound in state t, thereby forming a plurality of states for the experience.

In some implementations, satisfaction of the compound exit criterion is dependent on the type of score calculated. For instance, when the score is an absolute count of interaction features causal for binding, as disclosed in International Patent Application No. PCT/US24/24456, entitled “Systems and Methods for Discovering Compounds Using Causal Inference,” filed Apr. 12, 2024, which is hereby incorporated by reference, the initial compound in state t fails to satisfy the compound exit criterion when the absolute count is less than a threshold number of interaction features deemed to be sufficient for potent binding (e.g., less than 100, less than 50, less than 20, less than 10, etc.).

In some embodiments, the compound exit criterion is determined based on a predetermined hypothesis or prior.

In some embodiments, the compound exit criterion is determined based on one or more predetermined parameters known to be associated, highly causal, or necessary with a particular property relevant to interaction (e.g., binding potency, selectivity, ADME properties, toxicity, etc.). Predetermined parameters can be obtained from literature, published data, and/or experimental results. For instance, in some implementations, cutoff thresholds for ADME properties are determined based on outcomes of historical data on other molecules.

In some embodiments, the compound exit criterion is determined based on one or more parameters for a control molecule known to exhibit target properties. For instance, in some implementations, the compound exit criterion is determined by identifying one or more lead candidates or tool compounds that have been observed to exhibit target levels of binding, such as ADME properties, and/or drug-likeness. A lead candidate or tool compound is scored, using any one or more of the scoring methods disclosed above. The values obtained from the scoring methods are then used as a baseline threshold to establish the compound exit criterion for further assessment of other compounds. In some embodiments, a value obtained for a lead compound or tool compound is used to establish the compound exit criterion without alteration. Alternatively, in some embodiments, a value obtained for a lead compound or tool compound is used to adjust the compound exit criterion in order to establish the criterion value (e.g., to encourage identification of compounds having improved performance over the control compounds).

In some embodiments, the initial compound in state t is assigned a terminal positive reward when the compound exit criterion is satisfied.

In some embodiments, the initial compound in state t is assigned a terminal negative reward when the compound exit criterion is satisfied. In some embodiments, (ii), (iii), (iv), (v), (vi), (vii), and (viii) is repeated at least 2, at least 3, at least 4, at least 5, at least 6, at least 7, at least 8, or at least 9 times.

In some embodiments, a compound satisfies the compound exit criterion when the compound satisfies the requirements of Lipinski's Rule of Five, Veber's rules, the Ghose filter, the Egan filter, or Muegge's rule described in blocks 618-622 above.

Block 660. Referring to block 660, in some embodiments, the compound exit criterion is satisfied by either a negative condition of the initial compound in state t (e.g., the initial compound in state t exceeds a threshold molecular weight, exceeds a threshold total number of hydrogen bond donors, exceeds a threshold total number of hydrogen bond acceptors, exceeds a threshold number of aromatic rings, exceeds a threshold total polar surface area, etc.) or a positive condition of the initial compound in state t (e.g., achieves a score in 656 that satisfies a threshold condition, satisfies the requirements of Lipinski's Rule of Five, Veber's rules, the Ghose filter, the Egan filter, or Muegge's rule described in blocks 618-622 above, etc.). When the initial compound in state t has the positive condition, a terminal positive reward is assigned to the initial compound in state t and the (ix) repeating is optionally terminated. When the initial compound in state t has the negative condition, a terminal negative reward is assigned to the initial compound in state t and the (ix) repeating is optionally terminated.

Referring to block 408 of FIG. 4B, even in instances where a terminal condition has been reached for a given experience, the initial compound at state t=0 may be used in another experience. Since the molecular reaction and reactant at each state of the experience is separately sampled from probability distributions, the use of the same initial compound at state t=0 in several different instances will lead to different derived compounds 180. Accordingly, in some embodiments in accordance with block 408, the same selected initial compound (from state t=0) is used in 2, 3, 4, 5, 6, 7, 8, 9, 10, 11, 12, 13, 14, 15 or more, 20 or more, 25 or more, 50 or more, or 100 or more different experiences resulting in 2, 3, 4, 5, 6, 7, 8, 9, 10, 11, 12, 13, 14, 15 or more, 20 or more, 25 or more, 50 or more, or 100 or more different derived compounds 180. Thus, according to block 408 of FIG. 4B, if the selected initial compound (of state t=0) has been used in less a threshold number of different experiences, a new experience at a new state t=0 begins and process control returns to block 656 of FIG. 4B to reselect a molecular reaction for the initial compound at state t=0. Process control jumps to block 646 in some such embodiments, because the probability distribution of the molecular reactions for the initial compound in state t=0 is already available from the prior experience using the same initial compound in state t=0.

On the other hand, if the selected initial compound (of state t=0) has been used in less a threshold number of different experiences, process control goes to block 410 of FIG. 4C. In accordance with block 410, a determination is made as to whether a sufficient number of experiences have been generated to update the parameters of the parent model and the child model. If not, process control returns to block 164 to begin a new experience with a new initial compound from the initial compound data store 158. If a sufficient number of experiences have been evaluated then the parameters of the parent and child model can be updated. To update the parent and child models what is needed is the initial compound in each of the states of the experience, the final derived compound, and some metric for the activity of each such compound against the target macromolecule. In some embodiments, the metric for the activity of each such compound against the target macromolecule is determined by one or more physics model 192 or other scores described in block 656 above.

Block 666. Referring to block 666, in some embodiments, the physics model 192 evaluates an interaction energy of a complex of the initial compound in state t, or the derived compound, interacting with the environment 154 of the target macromolecule 152 as further described in block 656 above.

Blocks 668-672. Referring to block 668, in some embodiments of block 656, the physics model 192 of block 656 evaluates an interaction energy of a complex of the initial compound in state t, or the derived compound, interacting with the environment 154 of the target macromolecule using quantum mechanics, molecular mechanics with explicit solvent, molecular mechanics with a continuum solvent, or a heuristic model. Such quantum mechanics, molecular mechanics with explicit solvent, molecular mechanics with a continuum solvent, and heuristic models are summarized in Boas and Harbury, 2007, “Potential energy functions for protein design.” Current Opinion in Structural Biology. 17: 199-204, which is hereby incorporated by reference.

In some embodiments the physics model 192 of block 656 evaluates an interaction energy of a complex of the initial compound in state t, or the derived compound, interacting with the environment 154 of the target macromolecule using a calculated potential energy surface (potential energy function) of the initial compound and the environment of the target macromolecule.

Referring to block 670, in some such embodiments, the potential energy surface is calculated by the physics model using a molecular mechanics algorithm. Such molecular mechanics algorithms make use of molecular mechanics (MM) force fields, which are empirical models that describe the potential energy surfaces of molecular systems by treating them as collections of atomic point masses. These point masses interact via non-bonded and valence (bond, angle, and torsion) terms, which are typically parametrized to reproduce quantum chemical conformational energetics and physical properties. Sec, for example, Takaba et al., “Machine-learned molecular mechanics force fields from large-scale quantum chemical data,” arXiv:2307.07085v4 [physics.chem-ph] 8 Dec. 2023; Davies et al., 2002, “Structure-based design of a potent purine-based cyclin-dependent kinase inhibitor, Nature structural biology 9(10), pp. 745-749; and Hagler, 2019, “Force field development phase ii: Relaxation of physics-based criteria . . . or inclusion of more rigorous physics into the representation of molecular energetics,” Journal of computer-aided molecular design, 33 (2): 205-264, each of which is hereby incorporated by reference. Example programs for implementing the physics model using a quantum mechanics algorithm include, but are not limited to GROMACS, AMBER, CHARMM, NAMD, Desmond, Large-scale Atomic/Molecular Massively Parallel Simulator (LAMMPS), and OpenMM. See, for example, Thompson et al., 2022, “LAMMPS—a flexible simulation tool for particle-based materials modeling at the atomic, meso, and continuum scales,” Comp Phys Comm 271 p. 10817, and Shirts, et al., 2017, “Lessons learned from comparing molecular dynamics engines on the SAMPL5 dataset,” J Comput Aided Mol Des. 31(1), pp. 147-161, each of which is hereby incorporated by reference.

Referring to block 672, alternatively, in some such embodiments, the potential energy surface is calculated by the physics model using a quantum mechanics algorithm. Examples of quantum mechanics algorithm include, but are not limited to quantum mechanics-cluster (QM-Cluster), quantum mechanics/molecular mechanics (QM/MM), and continuum solvation methods. One review of such quantum mechanics algorithm is Ryde and Soderhjelm, 2016, “Ligand-Binding Affinity Estimates Supported by Quantum-Mechanical Methods,” Chem. Rev. 116, pp. 5520-5566, which is hereby incorporated by reference. Example programs for implementing the physics model using a quantum mechanics algorithm, include, but are not limited to Gaussian, ORCA, NWChem, GAMESS, Jaguar, and Psi4. See, for example, Peng et al., 2016, “Massively Parallel Implementation of Explicitly Correlated Coupled-Cluster Singles and Doubles Using TiledArray Framework,” The Journal of Physical Chemistry A 120(51), pp. 10231-10244, which is hereby incorporated by reference.

Block 674. Referring to block 674, in some embodiments, the physics model 192 of block 656 evaluates the initial compound in state t, or the derived compound, interacting with the environment 154 of the target macromolecule against an interaction feature contract. As used herein, the term “interaction feature contract” comprise a listing of potential interaction features that can form between an initial compound in state t and a binding pocket, as described in further detail in block 656.

Nonlimiting examples of interaction features that can be found in the interaction feature contract include three-dimensional partial charges, three-dimensional pharmacophores, and/or molecular dynamics residue interaction time.

In some embodiments, an interaction feature in the interaction feature contract is selected from the group consisting of hydrophobic interactions, hydrophobic areas, aromatic ring members, hydrogen bond acceptors, hydrogen bond donors, hydrogen bond acceptor in an aromatic ring, negatively charged species, positively charged species, metal coordination, and/or halogen bonds. In some embodiments, a respective interaction feature is a pharmacophore, such as a three-dimensional pharmacophore.

Three-dimensional pharmacophores have been used to capture the nature and three-dimensional arrangement of chemical functionalities in ligands that are relevant for molecular interactions with target macromolecules. Besides chemical nature and spatial arrangement, three-dimensional pharmacophores can capture feature directionality, such as in the case of hydrogen bonds and aromatic interactions. Additionally, spatial tolerance and weight can be fine-tuned for each pharmacophore feature to adjust its size and importance in the three-dimensional pharmacophore. In order to describe the preferable shape of molecules in an environment of the target macromolecule (e.g., binding site), pharmacophore features are often combined with exclusion volume constraints (also referred to as excluded volume constraints). For instance, an exclusion volume constraint can consist of a set of spheres that represent the protein residues imposing a barrier for binding of potential ligands.

Various tools are available in the art for modeling pharmacophores for ligand-target interactions (complex of the initial compound in state t interacting with the environment of the target macromolecule), including but not limited to FLAP, Pharmer, LigandScout, Catalyst, MOE, PHASE, Pharao, UNITY, and/or Forge. Three-dimensional pharmacophore elucidation methods can be classified as feature-based, substructure pattern-based, or molecular field-based, depending on how the pharmacophore features are derived. Feature-based methods derive pharmacophore features by filtering for geometric descriptors that match the characteristics of molecular interactions. Pattern-based methods, such as those implemented in PHASE, LigandScout, and Catalyst, detect substructures for chemical features in molecules. For example, all hydroxyl groups are defined as hydrogen bond donors and acceptors. In contrast, molecular field-based methods such as FLAP and Forge sample the molecular surface of either ligand or macromolecular target with different chemical probes and calculate interaction energy maps which can be translated into pharmacophore features. An additional distinction between three-dimensional pharmacophore generation methods is based on the type of employed data. This could be a set of active ligands, structural data on the ligand in complex with its macromolecular target, and/or structural data of the macromolecular target alone. Pharmacophores are further described, for example, in Schaller et al., “Next generation 3D pharmacophore modeling,” WIRES Comput Mol Sci. 2020; 10(4); Jiang and Rizzo, “Pharmacophore-based similarity scoring for dock,” J Phys Chem B. 2015; 119(3):1083-1102; and Arthur et al., “Hierarchical graph representation of pharmacophore models,” Front Mol Biosci. 2020; 7:599059, each of which is hereby incorporated herein by reference in its entirety.

In some embodiments, a respective interaction feature includes one or more corresponding geometric representations and/or one or more attribute values. In some embodiments, the dimensionality and nature of the geometric representations and/or attribute values of interaction features are dependent on the type of interaction feature; that is, a corresponding measurement appropriate for the respective interaction feature, as will be apparent to one skilled in the art. For instance, in some embodiments, a geometric representation of a respective interaction feature is a set of coordinates that indicates the position of the respective interaction feature in three-dimensional space for a respective conformation of the complex formed between an initial compound in state t and the environment of the target macromolecule. In some embodiments, a geometric representation of a respective interaction feature is a direction vector that indicates the direction or orientation of the respective interaction feature in three-dimensional space for the respective conformation of the complex formed between the of the initial compound in state t and the environment of the target macromolecule.

As another example, in some embodiments, an attribute value for a partial charge is a non-integer charge value when measured in elementary charge units; in yet another example, in some implementations, an attribute value for an aromatic ring pharmacophore includes a radius r of the aromatic ring.

Alternatively or additionally, in some embodiments, an attribute value for a respective interaction feature is a similarity score that measures a difference or a distance between the respective interaction feature in a complex formed between an initial compound in state t and the environment of the target macromolecule and a corresponding interaction feature in a reference conformation.

Alternatively or additionally, in some embodiments, an attribute value for a respective interaction feature is an indication of a presence or absence of the respective interaction feature at a corresponding position in a respective conformation of a complex formed between the initial compound in state t and the environment of the target macromolecule. In some embodiments, a corresponding geometric representation and/or a corresponding attribute value for a respective interaction feature is represented in a multi-dimensional space; for instance, in some embodiments, an attribute value for a hydrophobic interaction feature is represented as (1, 0, 0).

Interaction features are further described, for example, in Jiang and Rizzo, “Pharmacophore-based similarity scoring for dock,” J Phys Chem B. 2015; 119(3):1083-1102; and Arthur et al., “Hierarchical graph representation of pharmacophore models,” Front Mol Biosci. 2020; 7:599059, each of which is hereby incorporated herein by reference in its entirety.

In some embodiments, one or more dimension reduction techniques are applied to one or more geometric representations and/or one or more attribute values for a respective interaction feature.

In some embodiments, a dimension reduction reduces the dimensionality of a respective interaction feature from a first number of dimensions to a second number of dimensions. In some implementations, the starting number of dimensions varies between interaction features (e.g., a first interaction feature in a plurality of interaction features has the same or different number of starting dimensions as a second interaction feature in the plurality of interaction features). In some embodiments, the second number of dimensions after dimension reduction is the same or different for each interaction feature in a plurality of interaction features. For example, in some implementations, each respective interaction feature in a plurality of interaction features has a dimensionality of 1 after transformation.

In some embodiments, the dimension reduction is a principal components algorithm, a random projection algorithm, an independent component analysis algorithm, a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, each of which is hereby incorporated by reference.

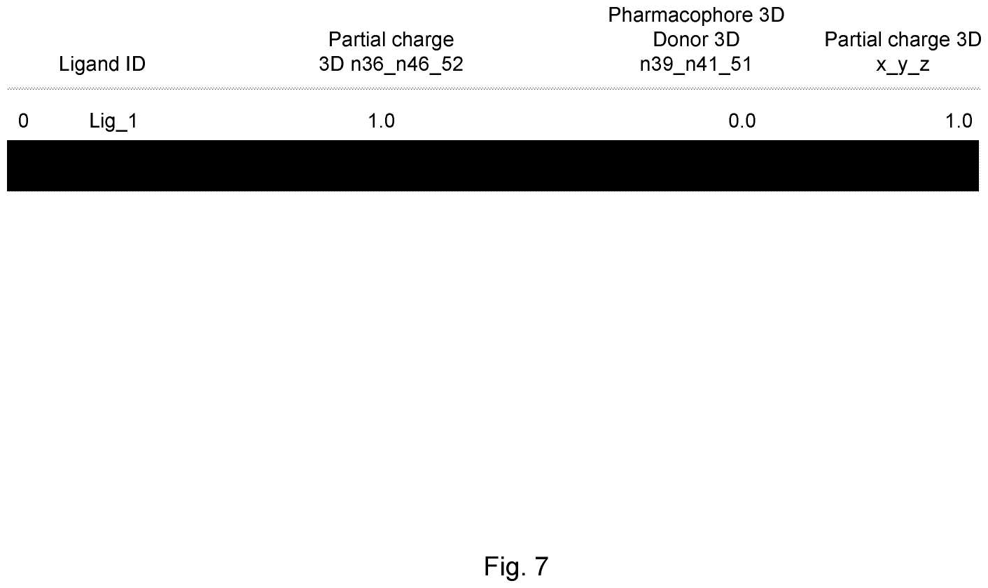

In some implementations, a geometric representation and/or an attribute value for a respective interaction feature is represented in scalar or binary values. In some implementations, upon application of a transformation to a respective interaction feature, the geometric representation and/or attribute value is further transformed from scalar values to binary values (e.g., 0 or 1). An example of an interaction feature vector for a corresponding candidate molecule, where the geometric representations and/or attribute values for each interaction feature in the interaction feature vector is binarized to zeros and ones, is illustrated in FIG. 7.

Block 676. Referring to block 676, in some embodiments, a derived compound 180 in the corresponding plurality of derived compounds requires at least two, at least three, or at least four different molecular reactions 212 in the plurality of molecular reactions to be synthesized from an initial compound in state t=0 used by the method to construct the derived compound.

In some embodiments, a derived compound 180 in the corresponding plurality of derived compounds requires at least 1, at least 2, at least 3, at least 4, at least 5, or at least 10 molecular reactions 212 in the plurality of molecular reactions to be synthesized from an initial compound in state t=0 used by the method to construct the derived compound. In some embodiments, a derived compound 180 in the corresponding plurality of derived compounds requires no more than 20, no more than 10, no more than 5, or no more than 2 molecular reactions 212 in the plurality of molecular reactions to be synthesized from an initial compound in state t=0 used by the method to construct the derived compound. In some embodiments, a derived compound 180 in the corresponding plurality of derived compounds requires from 1 to 5, from 2 to 10, or from 5 to 20 molecular reactions 212 in the plurality of molecular reactions to be synthesized from an initial compound in state t=0 used by the method to construct the derived compound. In some embodiments, a derived compound 180 in the corresponding plurality of derived compounds requires another range of molecular reactions 212, starting no lower than 1 molecular reaction and ending no higher than 20 molecular reactions, to be synthesized from an initial compound in state t=0 used by the method to construct the derived compound.

Block 678. Referring to block 678, in some embodiments, the complex of the initial compound in state t interacting with the environment 154 of the target macromolecule 152 comprises a plurality of poses (e.g., 2 or more poses, 10 or more poses, 100 or more poses, or 1000 or more poses) of the initial compound in state t docked into the environment of the target macromolecule. Further discussion of such poses is described in block 656, above, as well as the definitions section.

Block 680. Referring to block 680, in some embodiments, the plurality of molecular reactions that are evaluated by the parent model (e.g., in block 644 at a given state t) comprises 3, 4, 5, 6, 7, 8, 9, 10, 20, 30, 40, 50, 60, 70, 80, 90, or 100 or more molecular reactions.

Block 682. Referring to block 682, in some embodiments, the method further comprises masking those molecular reactions in the plurality of molecular reactions that are incompatible with an exit vector in an initial compound (e.g., before execution of block 644 for a given state t of a given experience 164). Such a filtering step improves computational efficiency of the parent model since fewer molecular reactions need to be evaluated by the parent model. This filtering step is illustrated as element 406 of FIG. 4A, in conjunction with block 406 above.

Block 684. Referring to block 684, in some embodiments, the plurality of experiences that are determined is twenty or more experiences representing 20 or more initial compounds in the plurality of initial compounds. In such an embodiment, when 20 experiences representing 20 initial compounds (e.g., from initial compound data store 156), process control in block 410 of FIG. 4C passes to block 686 and 690, discussed in further detail below, where the parent and child models are updated. Of course, the number 20 is given as just an example. Moreover, as further explained in block 408 above, any given compound 210 selected from the initial compound data store 156 to initiate one experience 164, may in fact be used in any number of other experiences as well. Thus, in some embodiments, while 20 experiences will likely represent 20 different derived compounds 180, it may represent fewer than 20 different compounds from the initial compound data store 156. In some embodiments, the plurality of experiences that are collected before turning process control to blocks 686 and 690 is more than 20, 30, 40, 50, 60, 70, 80, 90, or 100 experiences. In some embodiments, the plurality of experiences that are collected before turning process control to blocks 686 and 690 is more than 200, 300, 400, 500, 600, 700, 800, 900, or 1000 experiences. In some embodiments, the plurality of experiences that are collected before turning process control to blocks 686 and 690 is more than 2000, 3000, 4000, 5000, 6000, 7000, 8000, 9000, or 10,000 experiences. In some embodiments, the plurality of experiences that are collected before turning process control to blocks 686 and 690 is more than 20,000, 30,000, 40,000, 50,000, 60,000, 70,000, 80,000, 90,000, or 100,000 experiences. In some embodiments, the plurality of experiences that are collected before turning process control to blocks 686 and 690 is more than 1×10⁶, 1×10⁷, or 1×10⁸ experiences.

In some embodiments, the plurality of experiences that are collected before turning process control to blocks 686 and 690 represents more than 20, 30, 40, 50, 60, 70, 80, 90, or 100 different derived compounds. In some embodiments, the plurality of experiences that are collected before turning process control to blocks 686 and 690 represents more than 200, 300, 400, 500, 600, 700, 800, 900, or 1000 different derived compounds. In some embodiments, the plurality of experiences that are collected before turning process control to blocks 686 and 690 represents more than 2000, 3000, 4000, 5000, 6000, 7000, 8000, 9000, or 10,000 different derived compounds. In some embodiments, the plurality of experiences that are collected before turning process control to blocks 686 and 690 represents more than 20,000, 30,000, 40,000, 50,000, 60,000, 70,000, 80,000, 90,000, or 100,000 different derived compounds. In some embodiments, the plurality of experiences that are collected before turning process control to blocks 686 and 690 represents more than 1×10⁶, 1×10⁷, or 1×10⁸ different derived compounds.

### Update Parent Model 184.

Block 686. Referring to block 686, the first plurality of parameters 218-1, 218-2, . . . , 218-V, where V is a positive integer, of the parent model 184 is updated in accordance with a first surrogate objective 188 calculated using the plurality of experiences 164-1, 164-2, . . . , 164-M.

Referring to block 688, in some embodiments, the first surrogate objective 188 is a first trust region method. In some such embodiments, the first trust region method comprises:

$${maximize}_{θ} {\hat{𝔼}}_{t} [ \frac{{π}_{θ} ( {a}_{t} \vert {s}_{t} )}{{π}_{{θ}_{old}} ( {a}_{t} \vert {s}_{t} )} {\hat{A}}_{t} ]$$

$$subject to {\hat{𝔼}}_{t} [ K L [ {π}_{{θ}_{old}} ( \cdot \vert {s}_{t} ) , {π}_{θ} ( \cdot \vert {s}_{t} ) ] ] \leq δ$$

where,
   [symbol] ₜ is an empirical average taken over the plurality of states for an experience in the plurality of experiences by averaging

$$[ \frac{{π}_{θ} ( {a}_{t} \vert {s}_{t} )}{{π}_{{θ}_{old}} ( {a}_{t} \vert {s}_{t} )} {\hat{A}}_{t} ]$$

for each state t in the plurality of states for the experience,
θ_{old} is the first plurality of parameters prior to the updating of block 686,
θ is the first plurality of parameters upon performing the of block 686,
π_{θ}(aₜ|sₜ) is the probability assigned to each respective molecular reaction in the plurality of molecular reactions by the parent model for the complex of state t using θ,
π_{θ}_{_{old}}(aₜ|sₜ) is the probability assigned to each respective molecular reaction in the plurality of molecular reactions by the parent model at state t using θ_{old},
aₜ is the molecular reaction in the plurality of molecular reactions selected for state t,
sₜ is the initial compound in state t,

$${\hat{A}}_{t} = {δ}_{t} + ( γ λ ) {δ}_{t + 1} + … + … + {( γ λ )}^{T - t + 1} {δ}_{T - 1} ,$$

γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience,
KL[π_{θ}_{_{old}}(⋅|sₜ),π_{θ}(·|sₜ)] is a Kullback-Leibler (KL) divergence between the parent model with θ and the parent model with θ_{old}, and
δ is a maximum allowable KL divergence.

In some embodiments, δₜ has the form:

$${δ}_{t} = {r}_{t} + γ V ( {s}_{t + 1} ) - V ( {s}_{t} ) ,$$

rₜ is the score for state t,

$$V ( {s}_{t + 1} ) = {𝔼}_{π} [ {\sum }_{k = 0}^{∞} {γ}^{k} {r}_{t + 1 + k} \vert {s}_{t + 1} ] ,$$

$$V ( {s}_{t} ) = {𝔼}_{π} [ {\sum }_{k = 0}^{∞} {γ}^{k} {r}_{t + k} \vert {s}_{t} ] ,$$

rₜ₊₁₊ₖ is the score for state t+1+k, and
rₜ₊ₖ is the score for state t+k.

In some embodiments, the first trust region method updates θ_{old} to θ using an aggregate of [symbol] ₜ across each experience in the plurality of experiences. More details of such a trust region method are disclosed in Schulman et al., “Proximal Policy Optimization Algorithms,” arXiv:1707.06347v2 [cs.LG] 28 Aug. 2017, which is hereby incorporated by reference.

Referring to block 690, in some embodiments, the first surrogate objective 188 is a clipped surrogate objective. In some such embodiments, the clipped surrogate objective comprises:

$${L}^{CLIP} ( θ ) = {\hat{𝔼}}_{t} [ min ( {r}_{t} ( θ ) {\hat{A}}_{t} , clip ( {r}_{t} ( θ ) , 1 - ϵ , 1 + ϵ ) {\hat{A}}_{t} ) ]$$

where,
   [symbol] ₜ is an expectation taken over the plurality of states for an experience in the plurality of experiences,
   θ is the first plurality of parameters upon performing the updating of block 686,

$${r}_{t} ( θ ) = \frac{{π}_{θ} ( {a}_{t} \vert {s}_{t} )}{{π}_{{θ}_{old}} ( {a}_{t} \vert {s}_{t} )} ,$$

π_{θ}(aₜ|sₜ) is the probability assigned to each respective molecular reaction in the plurality of molecular reactions by the parent model for the complex of state t using θ,
π_{θ}_{_{old}}(aₜ|sₜ) is the probability assigned to each respective molecular reaction in the plurality of molecular reactions by the parent model at state t using θ_{old},

$${\hat{A}}_{t} = {δ}_{t} + ( γ λ ) {δ}_{t + 1} + … + … + {( γ λ )}^{T - t + 1} {δ}_{T - 1} ,$$

γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience, and
clip(rₜ(θ),1−ϵ,1+ϵ) is a clipped version of rₜ(θ) bounded within the range 1-ϵ, 1+ϵ.

In some embodiments, the clipped surrogate objective updates θ_{old} to θ using an aggregate of [symbol] ₜ across each experience in the plurality of experiences. More details of such a clipped surrogate objective are disclosed in Schulman et al., “Proximal Policy Optimization Algorithms,” arXiv:1707.06347v2 [cs.LG] 28 Aug. 2017, which is hereby incorporated by reference.

### Update Child Model 186.

Referring to block 690, the second plurality of parameters 220-1, 220-2, . . . , 220-W, where W is a positive integer, of the child model 186 is updated in accordance with a second surrogate objective 190 using the plurality of experiences 164-1, 164-2, . . . , 164-M. In some embodiments, the second surrogate objective 190 is a trust region method or a clipped surrogate objective analogous to that applied for the first surrogate objective 188, such as one of the objectives disclosed in Schulman et al., “Proximal Policy Optimization Algorithms,” arXiv:1707.06347v2 [cs.LG] 28 Aug. 2017, which is hereby incorporated by reference.

### Repeat Until a Threshold Convergence Criterion is Satisfied.

Referring to block 693, the generating 612, updating 686, and updating 690, is repeated until a threshold convergence criterion is satisfied. In some embodiments, the generating 612, updating 686, and updating 690 is repeated at least 2, at least 3, at least 4, at least 5, at least 10, at least 20, at least 50, or at least 100 times using at least 2, at least 3, at least 4, at least 5, at least 10, at least 20, at least 50, or at least 100 different initial compounds thereby deriving at least 2, at least 3, at least 4, at least 5, at least 10, at least 20, at least 50, or at least 100 derived compounds. In some embodiments, the generating 612, updating 686, and updating 690 is repeated no more than 200, no more than 100, no more than 50, no more than 10, or no more than 5 times until a threshold convergence criterion is satisfied. In some embodiments, the generating 612, updating 686, and updating 690 is repeated from 2 to 10, from 5 to 50, from 30 to 100, or from 100 to 200 times until a threshold convergence criterion is satisfied. In some embodiments, the generating 612, updating 686, and updating 690 is repeated is repeated a number of times that falls within another range starting no lower than 2 times and ending no higher than 1×10¹⁰ times prior to satisfying a threshold convergence criterion.

In some embodiments, the threshold convergence criterion is a gradient norm threshold. In such embodiments the threshold convergence criterion is satisfied when the norm of a gradient of the objective function (e.g., expected reward) of the parent model with respect to parent model parameters and/or the child model with respect to the child model parameters falls below a predefined threshold (e.g., 10⁻³ or 10⁻⁴) indicating that changes to the first plurality of parameters of the parent model are becoming negligible, suggesting that the policy is approaching a local optimum.

In some embodiments, the threshold convergence criterion is an improvement in expected reward in which the threshold convergence criterion is satisfied when the improvement in the expected reward for the parent model and/or child model over a certain number of iterations (412—No of FIG. 4) is below a specified threshold. This can be measured by average the expected reward of the parent model and/or child model over recent episodes (e.g., each instance of 412—No of FIG. 4 is an example of beginning a new episode). In some such embodiments, a difference of ϵ=10-2 or lower, over a set number of episodes (e.g., 2, 3, 4, 5, 6, 7, 8, 9, 10) is a suitable threshold.

In some embodiments, the threshold convergence criterion is a maximum number of iterations (412—No of FIG. 4). For instance in some embodiments, the threshold convergence criterion is satisfied when the generating 612, updating 686, and updating 690 has been repeated 2, 3, 4, 5, 10, 20, 50, or 100 times. In some embodiments, the threshold convergence criterion is satisfied when the generating 612, updating 686, and updating 690 has been repeated 200, 100, 50, 10, or 5 times. In some embodiments, the threshold convergence criterion is satisfied when the generating 612, updating 686, and updating 690 has been repeated between 2 to 10, between 5 to 50, between 30 to 100, or between 100 to 200 times. In some embodiments, the threshold convergence criterion is satisfied when the generating 612, updating 686, and updating 690 has been repeated a number of times that falls within another range starting no lower than 2 times and ending no higher than 1×10¹⁰ times.

In some embodiments, the threshold convergence criterion is a metric for policy stability (e.g., the stability of the first and/or second plurality of parameters) under which the threshold convergence criterion is satisfied when a divergence between successive policies (e.g., divergence between the first and/or second plurality of parameters in successive repetitions of the generating 612, updating 686, and updating 690 (e.g., measured using a distance metric like KL-divergence) becomes small (e.g., a KL-divergence of less than 0.01).

### Test a Subset of the Plurality of Derived Compounds.

Block 694. Referring to block 694, a subset of the plurality of derived compounds 180, from the plurality of experiences, are tested in an assay (e.g., a wet lab assay) for activity against the target macromolecule, thereby identifying one or more derived compounds that exhibit the threshold activity with respect to the target macromolecule. In some embodiments, the subset of the plurality of derived compounds is 2, 3, 4, 5, 6, 7, 8, 9, or 10 or more derived compounds. In some embodiments, the subset of the plurality of derived compounds is at least 20, 30, 40, 50, 60, 70, 80, 90, or 100 derived compounds. In some embodiments, the subset of the plurality of derived compounds is at least 200, 300, 400, 500, 600, 700, 800, 900, or 1000 derived compounds. In some embodiments, the subset of the plurality of derived compounds is between 5 and 1000, 10 and 2000, or 20 and 3000 derived compounds. In some embodiments, the subset of the plurality of derived compounds is more than two derived compounds and less than 100, 500, or 1000 derived compounds.

In some embodiments, derived compounds, or initial compounds in state t in any stage of the described processes are validated using a molecular dynamics simulation of the compound interacting the environment of the target macromolecule. Molecular dynamics simulations capture the behavior of proteins and other biomolecules in full atomic detail and at very fine temporal resolution. Such simulations can be used to decipher the functional mechanisms of proteins and other biomolecules, uncover the structural basis for disease, and aid in the design and optimization of small molecules, peptides, and proteins. See, for example, Durrant and McCammon, “Molecular dynamics simulations and drug discovery,” BMC Biology. 2011; 9(1):71; and Hollingsworth and Dror, “Molecular dynamics simulation for all,” Neuron. 2018; 99(6):1129-1143, each of which is hereby incorporated herein by reference in its entirety.

Block 696. Referring to block 696, in some embodiments the threshold activity with respect to the target macromolecule is an IC₅₀, EC₅₀, K_{d}, K_{I}, hill coefficient (nH), negative logarithm of EC₅₀ (pEC50), association rate constant (Kon), or disassociation rate constant (Koff), for a derived compound with respect to the target macromolecule. Accordingly, in some embodiments, one or more derived compounds identified using the systems and methods of the present disclosure are synthesized and tested in a wet lab assay to determine whether they have potency against a therapeutic target. In some embodiments, the goal of such an assay is to determine a binding coefficient of the compound to a target macromolecule. In some such embodiments, the binding coefficient is an IC₅₀, EC₅₀, Kd, KI, or pKI for the compound with respect to the target macromolecule.

In some embodiments a derived compound has a threshold activity with respect to the target macromolecule when the derived compound has an IC₅₀, EC₅₀, Kd, or KI of less than 1 molar, less than 1 millimolar, less than 100 micromolar, less than 10 micromolar, less than 1 micromolar, less than 100 nanomolar, less than 10 nanomolar, or less than 1 nanomolar.

In some embodiments, the target macromolecule is associated with a condition. In some embodiments, the condition is a disease. In some embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

In some embodiments the wet lab assay test validates a compound identified by the systems and methods of the present disclosure as being a suitable compound for alleviation of the condition. In some such embodiments the compound is used in in vivo assays such as animal models.

In some embodiments, a compound identified by the systems and methods of the present disclosure is combined with one or more excipient and/or one or more pharmaceutically acceptable carrier and/or one or more diluent when administering to an animal model or a human.

Such excipients and/or carriers include all conventional solvents, dispersion media, fillers, solid carriers, coatings, antifungal and antibacterial agents, dermal penetration agents, surfactants, isotonic and absorption agents and the like.

An exemplary carrier is pharmaceutically “acceptable” in the sense of being compatible with the other ingredients of the composition (e.g., the composition comprising the selected compound in the plurality of compounds) and not injurious to a subject. The compound may conveniently be presented in unit dosage form and may be prepared by any of the methods well known in the art of pharmacy. Such methods include bringing into association the compound with the carrier that constitutes one or more accessory ingredients. In general, the compound is prepared by uniformly and intimately bringing into association the compound with liquid carriers or finely divided solid carriers or both.

Exemplary compounds formulated for intravenous, intramuscular or intraperitoneal administration, or a pharmaceutically acceptable salt, solvate or prodrug thereof may be administered by injection or infusion.

In some embodiments, injectables for such use are prepared in conventional forms, either as a liquid solution or suspension or in a solid form suitable for preparation as a solution or suspension in a liquid prior to injection, or as an emulsion. In some embodiments, carriers include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

In some embodiments, the compound (e.g., derived compound) is also suitable for oral administration and presented as discrete units such as capsules, sachets or tablets each containing a predetermined amount of the test chemical compound; as a powder or granules; as a solution or a suspension in an aqueous or non-aqueous liquid; or as an oil-in-water liquid emulsion or a water-in-oil liquid emulsion. In some embodiments, the compound (e.g., derived compound) is presented as a bolus, electuary or paste.

In some embodiments, a tablet of the compound is made by compression or molding, optionally with one or more accessory ingredients. In some embodiments, compressed tablets are prepared by compressing in a suitable machine the test chemical compound in a free-flowing form such as a powder or granules, optionally mixed with a binder [e.g., inert diluent, preservative disintegrant (e.g. sodium starch glycolate, cross-linked polyvinyl pyrrolidone, cross-linked sodium carboxymethyl cellulose) surface-active or dispersing agent]. In some embodiments, molded tablets are made by molding in a suitable machine a mixture of the powdered compound moistened with an inert liquid diluent. In some embodiments, the tablets are optionally coated or scored and may be formulated so as to provide slow or controlled release of the compound therein using, for example, hydroxypropylmethyl cellulose in varying proportions to provide the desired release profile. In some embodiments, tablets are optionally provided with an enteric coating, to provide release in parts of the gut other than the stomach.

In some embodiments, the compound (e.g., derived compound) is suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

In some embodiments, the compound (e.g., derived compound) is suitable for topical administration to the skin. In some such instances, the compound is dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. In some embodiments, transdermal patches are used to administer the compound.

In some embodiments, the compound (e.g., derived compound) is suitable for parenteral administration. In such embodiments, the compound includes aqueous and non-aqueous isotonic sterile injection solutions that contain anti-oxidants, buffers, bactericides and solutes that render the compound isotonic with the blood of the intended recipient; and aqueous and non-aqueous sterile suspensions that include suspending agents and thickening agents. In some embodiments, the compound is presented in unit-dose or multi-dose sealed containers, for example, ampoules and vials, and stored in a freeze-dried (lyophilized) condition requiring only the addition of the sterile liquid carrier, for example water for injections, immediately prior to use. In some embodiments, extemporaneous injection solutions and suspensions are prepared from sterile powders, granules and tablets of the kind previously described.

It should be understood that in addition to the compound particularly mentioned above (e.g., derived compound), the composition or combination of this present disclosure (e.g., the selected derived compound) may include other agents conventional in the art having regard to the type of composition or combination in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In some embodiments, the present disclosure informs the selection of one or more human subjects for treatment with the compound (e.g., derived compound) and/or selection of one or more human subjects for continuation or discontinuation of treatment with the compound.

In some embodiments, the present disclosure informs the dosing amount, duration, and/or frequency of the compound in one or more human subjects for treatment.

In some embodiments, the present disclosure informs the design of a clinical trial, the clinical trial comprising the use of the compound (e.g., derived compound). In some embodiments, the present disclosure informs the design of an adaptive clinical trial, the adaptive clinical trial comprising the use of the compound.

In some embodiments, the present disclosure further comprises formulating the compound (e.g., derived compound) for use in a therapy. In some embodiments, this includes formulating the compound with any of the excipients, pharmaceutically acceptable carrier, diluents, or other pharmacological formulations described in the present disclosure or known in the art. In some embodiments, the therapy is to alleviate a condition such as inflammation. In some embodiments the therapy is to alleviate or treat a disease or disorder. In some embodiments the disease or disorder is cancer, a hematologic disorder, an autoimmune disease, an inflammatory disease, an immunological disorder, a metabolic disorder, a neurological disorder, a genetic disorder, a psychiatric disorder, a gastroenterological disorder, a renal disorder, a cardiovascular disorder, a dermatological disorder, a respiratory disorder, a viral infection, or other disease or disorder.

Use cases. In some embodiments, the systems and methods disclosed herein are advantageously used in any number of applications, including but not limited to hit discovery, hit-to-lead discovery, lead optimization, off-target side-effect prediction, molecular dynamics simulations, toxicity prediction, potency optimization, selectivity optimization, fitness modeling, drug repurposing, drug resistance prediction, personalized medicine, drug trial design, agrochemical design, and/or materials science.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method for identifying one or more derived compounds that exhibit a threshold activity with respect to a target macromolecule, using a plurality of initial compounds, the method comprising:
A) generating, using a computer, a plurality of experiences, each respective experience in the plurality of experiences using an initial compound selected from the plurality of initial compounds to construct a corresponding derived compound through a hierarchical proximal policy comprising a parent model and a child model using an environment of the target macromolecule, thereby generating a plurality of derived compounds, wherein
the parent model is a molecular reaction model that evaluates a plurality of molecular reactions,
the child model is a reactant model that evaluates a corresponding plurality of reactants for a molecular reaction,
the parent model comprises a first plurality of parameters, and
the child model comprises a second plurality of parameters,
B) updating, using a computer, the first plurality of parameters in accordance with a first surrogate objective calculated using the plurality of experiences;
C) updating, using a computer, the second plurality of parameters in accordance with a second surrogate objective using the plurality of experiences;
D) repeating, using a computer, the generating A), updating B), and updating C) until a threshold convergence criterion is satisfied; and
E) testing a subset of the plurality of derived compounds, from the plurality of experiences, in a wet lab assay for activity against the target macromolecule, thereby identifying one or more derived compounds that exhibit the threshold activity with respect to the target macromolecule.

2. The method of claim 1, wherein an experience in the plurality of experiences is generated by:
(i) initializing the experience to state t=0,
(ii) inputting a complex of state t, in two or three dimensions, of the initial compound in state t interacting with the environment of the target macromolecule into the parent model, wherein the parent model evaluates, using a computer, a first exit vector of the initial compound in state t against the plurality of molecular reactions, thereby assigning a corresponding probability to each respective molecular reaction in the plurality of molecular reactions for state t,
(iii) selecting a molecular reaction in the plurality of molecular reactions, using a computer, through a sampling of the plurality of molecular reactions using the corresponding probability assigned to each molecular reaction in the plurality of molecular reactions for state t,
(iv) inputting the complex of state t into the child model, wherein the child model evaluates, using a computer, the initial compound in state t against each reactant in a corresponding plurality of reactants available for reaction using the molecular reaction selected for state t, thereby assigning a corresponding probability to each respective reactant in the corresponding plurality of reactants for state t,
(v) selecting, using a computer, a reactant in the corresponding plurality of reactants, through a sampling of the corresponding plurality of reactants using the corresponding probability assigned to each reactant in the corresponding plurality of reactants for state t,
(vi) advancing state t to state t+1,
(vii) forming, using a computer, the initial compound in state t through an in silico reaction of the initial compound in state t−1 in accordance with the selected molecular reaction and the selected reactant of state t,
(viii) determining a score, using a computer, for the initial compound in state t interacting with the environment of the target macromolecule by inputting the initial compound in state t interacting with the environment of the target macromolecule into a physics model, and
(ix) repeating the (ii) inputting, (iii) selecting, (iv) inputting, (v) selecting, (vi) advancing, (vii) forming, and (viii) determining until a compound exit criterion is satisfied by the initial compound in state, thereby forming a plurality of states for the experience.

3. The method of claim 1, wherein the plurality of molecular reactions comprises twenty or more molecular reactions.

4. (canceled)

5. The method of claim 1, wherein the method further comprises masking those reactions in the plurality of molecular reactions that are incompatible with an exit vector in an initial compound.

6. The method of claim 1, wherein the corresponding plurality of reactants comprises twenty or more reactants.

7. The method of claim 1, wherein the plurality of experiences is twenty or more experiences representing 20 or more initial compounds in the plurality of initial compounds.

8. The method of claim 2, wherein the first surrogate objective is a first trust region method.

9. The method of claim 8, wherein the first trust region method comprises:
$${maximize}_{θ} {\hat{𝔼}}_{t} [ \frac{{π}_{θ} ( {a}_{t} \vert {s}_{t} )}{{π}_{{θ}_{old}} ( {a}_{t} \vert {s}_{t} )} {\hat{A}}_{t} ]$$
$$subject to {\hat{𝔼}}_{t} [ K L [ {π}_{{θ}_{old}} ( \cdot \vert {s}_{t} ) , {π}_{θ} ( \cdot \vert {s}_{t} ) ] ] \leq δ$$
wherein, [symbol]
ₜ is an empirical average taken over the plurality of states for an experience in the plurality of experiences by averaging
$$[ \frac{{π}_{θ} ( {a}_{t} \vert {s}_{t} )}{{π}_{{θ}_{old}} ( {a}_{t} \vert {s}_{t} )} {\hat{A}}_{t} ]$$
for each state t in the plurality of states for the experience,
θ_{old} is the first plurality of parameters prior to the updating B),
θ is the first plurality of parameters upon performing the updating B),
π_{θ}(aₜ|sₜ) is the probability assigned to each respective molecular reaction in the plurality of molecular reactions by the parent model for the complex of state t using θ,
π_{θ}_{_{old}}(aₜ|sₜ) is the probability assigned to each respective molecular reaction in the plurality of molecular reactions by the parent model at state t using θ_{old},
aₜ is the molecular reaction in the plurality of molecular reactions selected for state t,
sₜ is the initial compound in state t,
$${\hat{A}}_{t} = {δ}_{t} + ( γ λ ) {δ}_{t + 1} + … + … + {( γ λ )}^{T - t + 1} {δ}_{T - 1} ,$$
γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience,
KL[π_{θ}_{_{old}}(⋅|sₜ),π_{θ}(⋅|sₜ)] is a Kullback-Leibler (KL) divergence between the parent model with θ and the parent model with θ_{old}, and
δ is a maximum allowable KL divergence.

10. The method of claim 9, wherein δₜ has the form:
$${δ}_{t} = {r}_{t} + γ V ( {s}_{t + 1} ) - V ( {s}_{t} ) , wherein$$
$${r}_{t} is the score for state t ,$$
$$V ( {s}_{t + 1} ) = {𝔼}_{π} [ {\sum }_{k = 0}^{∞} {γ}^{k} {r}_{t + 1 + k} \vert {s}_{t + 1} ] ,$$
$$V ( {s}_{t} ) = {𝔼}_{π} [ {\sum }_{k = 0}^{∞} {γ}^{k} {r}_{t + k} \vert {s}_{t} ] ,$$
$${r}_{t + 1 + k} is the score for state t + 1 + k , and$$
$${r}_{t + k} is the score for state t + k .$$

11. The method of claim 9, wherein the first trust region method updates θ_{old} to θ using an aggregate of [symbol] ₜ across each experience in the plurality of experiences.

12. The method of claim 2, wherein the surrogate objective is a clipped surrogate objective.

13. The method of claim 12, wherein the clipped surrogate objective comprises:
$${L}^{CLIP} ( θ ) = {\hat{𝔼}}_{t} [ min ( {r}_{t} ( θ ) {\hat{A}}_{t} , clip ( {r}_{t} ( θ ) , 1 - ϵ , 1 + ϵ ) {\hat{A}}_{t} ) ]$$ [symbol]
ₜ is an expectation taken over the plurality of states for an experience in the plurality of experiences,
θ is the first plurality of parameters upon performing the updating B),
$${r}_{t} ( θ ) = \frac{{π}_{θ} ( {a}_{t} \vert {s}_{t} )}{{π}_{{θ}_{old}} ( {a}_{t} \vert {s}_{t} )} ,$$
π_{θ}(aₜ|sₜ) is the probability assigned to each respective molecular reaction in the plurality of molecular reactions by the parent model for the complex of state t using θ,
π_{θ}_{_{old}} (aₜ|sₜ) is the probability assigned to each respective molecular reaction in the plurality of molecular reactions by the parent model at state t using θ_{old},
$${\hat{A}}_{t} = {δ}_{t} + ( γ λ ) {δ}_{t + 1} + … + … + {( γ λ )}^{T - t + 1} {δ}_{T - 1} ,$$
γ is a scalar between 0 and 1,
λ is a smoothing parameter,
δₜ is a temporal difference error at state t that represents a difference between (i) a predicted score for the initial compound in state t (ii) and the actual score for the initial compound in state t, plus an estimated score for the initial compound in state t+1,
T is the number of states in the experience, and
clip(rₜ(θ),1-ϵ, 1+ϵ) is a clipped version of rₜ(θ) bounded within the range 1-ϵ, 1+ϵ.

14. The method of claim 13, wherein the clipped surrogate objective updates θ_{old} to θ using an aggregate of [symbol] ₜ across each experience in the plurality of experiences.

15. The method of claim 1, wherein the first plurality of parameters comprises at least 10,000, at least 100,000, or at least 1×10⁶ parameters.

16-20. (canceled)

21. The method of claim 1, wherein each initial compound in the plurality of initial compounds is an organic compound having a molecular weight of between 500 Daltons and 1000 Daltons.

22. The method of claim 1, wherein each derived compound in the plurality of derived compounds is an organic compound having a molecular weight of between 400 Daltons and 10000 Daltons.

23-25. (canceled)

26. The method of claim 1, wherein a derived compound in the plurality of derived compounds requires at least two different molecular reactions in the plurality of molecular reactions to be synthesized from an initial compound used by the method to construct the derived compound.

27-32. (canceled)

33. The method of claim 1, wherein the compound exit criterion is satisfied by either a negative condition of the initial compound in state t or a positive condition of the compound in state t, wherein,
when the initial compound in state t has the positive condition, a terminal positive reward is assigned to the initial compound in state t and the (ix) repeating is terminated, and
when the initial compound in state t has the negative condition, a terminal negative reward is assigned to the initial compound in state t and the (ix) repeating is terminated.

34. The method of claim 1, wherein the parent model is a first graph neural network, wherein the first graph neural network is a first graph isomorphism neural network.

35. (canceled)

36. The method of claim 1, wherein the child model is a second graph neural network that is passed an output of the parent model, wherein the second graph neural network is a second graph isomorphism neural network.

37-48. (canceled)
